(19) United States
(12) Patent Application Publication
(10) Pub. No.: US 2013/0134168 A1
(43) Pub. Date: May 30, 2013

(54) SOLUBILIZED COLLAGEN FIBER BALL

(71) Applicants: Tomohiko Yaguchi, Yamagata-shi (JP); Shingo Nakazaki, Yamagata-shi (JP); Junko Suzuki, Yamagata-shi (JP)

(72) Inventors: Tomohiko Yaguchi, Yamagata-shi (JP); Shingo Nakazaki, Yamagata-shi (JP); Junko Suzuki, Yamagata-shi (JP)

(73) Assignee: MIDORI HOKUYO CO., LTD., Yamagata-shi, Yamagata (JP)

(21) Appl. No.: 13/700,607

(22) PCT Filed: May 30, 2011
(86) PCT No.: PCT/JP2011/062865

§ 371 Date: Nov. 28, 2012

(30) Foreign Application Priority Data
May 28, 2010 (JP) 2010-123091

(51) Int. Cl. A61K 8/02; B65D 25/04

(52) U.S. Cl. CPC A61K 8/027; B65D 25/04
USPC 220/500; 424/401

(57) ABSTRACT

Disclosed is solubilized collagen fiber ball: that is crimped and does not have fiber separation defects within which the amount of water content and the amount of hydrophilic organic solvent are evenly distributed; and that dissolve instantly and uniformly when used. The fiber ball has a composition that may comprise 66 to 87 wt % solubilized collagen, 2 to 6 wt % buffer salt, 10 to 22 wt % water content, and a trace amount to 6 wt % residual hydrophilic organic solvent, and is 3 to 25 mm in diameter, weighs 3 to 20 mg, has an average fiber size of 3 to 10 dtx, and a length of 1 to 20 cm. The fiber ball, which has a wavy shape and comprises uniform bundles of solubilized collagen fibers formed in a spherical shape, may have a bulk density of 4.0 to 8.0 mg/cm3, and a swab diameter of 3 to 25 mm.

### TECHNICAL FIELD

The present invention relates to a solubilized collagen fiber ball.

### BACKGROUND ART

A product composed of insolubilized collagen fibers, obtained by first producing solubilized fibers from atelocollagen by decomposing an insoluble collagen and then regenerating the collagen as a solubilized collagen, and subsequently performing crosslinking to achieve insolubilization is already known (Patent Document 1: JP 07-83759 B).

Generally, products that are called collagen fibers refer to fibrous materials formed from insolubilized collagen fibers. By performing this insolubilization, when the product is used as a medical material, the properties of the product include good strength and insolubility in water. Further, also known are a method for producing a continuous collagen single fiber for medical purposes by dewatering and solidifying a collagen solution in a hydrophilic organic solvent having a water content of 10% or less, subsequently drying the solidified collagen under conditions including a relative humidity of 50% or less and a temperature of 42° C. or lower while maintaining tension on the fiber, and then supplying the fiber to a crosslinking reaction treatment (Patent Document 2: JP 4,356,654 B), and a method for drying a swollen-state porcine-derived type I, type-III mixed collagen thread-like material, wherein by passing the thread-like material to be dried through an air duct in the axial direction, while also applying a revolving air flow that travels through the air duct with a helical revolving motion, the thread-like material undergoes a revolving motion similar to a skipping rope (Patent Document 3: JP 4,000,855 B).

Solubilized collagen fibers used as cosmetics usually exist in a fibrous state containing mainly a solubilized collagen and water. Conventionally, storage of aqueous solutions of solubilized collagen has been considered difficult. Accordingly, it was thought that products that could be stored as solubilized collagen fibers, and then dissolved in an aqueous solution at the time of use to form a solubilized collagen aqueous solution would be very convenient for people who use solubilized collagen aqueous solutions as cosmetics. However, the production of solubilized collagen fibers has been found to be problematic. As a result of much research, the inventors of the present invention succeeded in producing soluble solubilized collagen fibers by shortening the length of the fibers, but they were unable to obtain a product that could be used as a cosmetic material. As a result of further research, the inventors succeeded in producing solubilized collagen fibers, but because they were unable to obtain solubilized collagen fibers in which uniformity was maintained throughout the entire fiber, the product was not totally satisfactory as a cosmetic material.

It was found that by applying a solubilized collagen aqueous solution obtained by dissolving solubilized collagen fibers in water to the surface of the skin, the action of the solubilized collagen was able to impart moisture to the skin. This should enable the development of new types of cosmetics, and is attracting much attention. In conventional products, dissolving the solubilized collagen fibers instantly and uniformly in water to obtain a solubilized collagen aqueous solution has proven difficult, and the development of solubilized collagen fibers that can be used as cosmetics remains a problem that requires addressing.

Details relating to the development of solubilized collagen fibers by the inventors of the present invention are presented below.

In an invention relating to solubilized collagen fibers proposed by the inventors of the present invention (Patent Document 4: JP 2005-306736 A, JP 4,401,226 B), the solubility of the fibers was improved by shortening the fiber length, but because the obtained solubilized collagen fibers were not sufficiently fine, the speed of the dissolution in water was not entirely satisfactory for use as a cosmetic. Subsequently, the inventors of the present invention pursued research into enabling the continuous production of solubilized collagen fibers for cosmetics under stable operating conditions.

Solubilized collagen fibers proposed by the inventors of the present invention (Patent Document 5: JP 2006-342472 A, JP 4,628,191 B) were obtained by preparing a raw material solution of a solubilized collagen having an isoionic point of pH 5.0 or less, discharging the solution from a nozzle into isopropyl alcohol in the same manner as that described above, thus forming a fiber bundle of solubilized collagen fibers for cosmetics, performing spinning and drawing of the fibers, immersing the fibers in a hydrophilic organic solvent, and then removing the water content and organic solvent from the fibers. In the research for this invention, continuous roller drying was performed in which drying was performed by passing the solubilized collagen fiber bundle continuously through a winding device 21 while air was blown across the fibers (FIG. 2, left). It was discovered that, in this case, performing continuous roller drying under an air flow caused tension to be applied to the fibers, meaning crimping of the collagen fibers did not occur, drying tended to occur with the fibers adhered to one another, resulting in fiber separation defects (adhesion and agglutination of the fiber bundle), and the removal of the water content and organic solvent was also unsatisfactory, resulting in poor drying efficiency. In summary, a stable drying operation could not be achieved. Accordingly, this method was abandoned.

Because satisfactory results could not be achieved using a continuous drying method, a batch drying method was adopted in which the solubilized collagen fiber bundle was suspended inside a clean bench and air was then blown across the fibers (this batched hanging drying is illustrated in FIG. 2, right). In this case, because the individual fibers separated to some extent and were moved by the air flow, crimping was able to be achieved to some extent, and an improvement in fiber separation was also observed. However, the water content and the organic solvent tended to move downward during the drying, meaning non-uniformity in the water content and organic solvent content was observed in portions of the fibers, and because this problem was unavoidable, solubilized collagen fibers that were uniform throughout the entire fiber were unobtainable. As a result, agglutination occurred in portions of the solubilized collagen fiber bundle.

The fiber bundle was placed between two drums each having a plurality of wires embedded therein, and a fiber opening (where the fiber bundle is separated into individual fibers) was performed by rotating the drums so that the wires did not make contact with each other, thus forming a fibrous state. Satisfactory fiber opening could not be achieved, and the drying treatment was inadequate, and those portions where the fibers had undergone agglutination were cut away and not used. At the time of packaging, the solubilized collagen fibers were converted to a rounded fibrous state, and subsequently collected and packaged. The thus obtained solubilized collagen fibers had a fineness of 10 dtx (the number of grams per 10,000 m of fiber) or less, and were able to be dissolved uniformly in water within 30 seconds. As a result, the inventors of the present invention were able to complete the above invention. In this invention, it is reported that in order to enable use of the solubilized collagen fibers as a cosmetic, it is desirable to form the fibers in a soft and expanded fibrous state (see 0034 to 0038, and 0074 to 0080 of the aforementioned JP 2006-342472 A).

As stated above, the fibrous state solubilized collagen fibers described above include non-uniform portions. It is necessary to ensure that the fiber components exist in a uniform state throughout the fibers. Further, in the method described above, even when the fibers are used as a cosmetic, the solubilized collagen fibers are accumulated in an irregular manner. It is thought that for these reasons, when the aforementioned fibrous solubilized collagen fibers are dissolved in water, unsatisfactory dissolution tends to result.

Furthermore, in terms of the collagen fiber bundle obtained following the drying step, which represents one portion of the production process, various problems are noticeable, including the fact that crimping has not been applied in some portions, the existence of fiber separation defects (adhesion of the fiber bundle) in some portions, and the existence of portions in which the removal of the water content and the organic solvent is unsatisfactory, resulting in agglutination of the fibers, and it was clear that it was necessary to ensure that the structure of the fibers enabled instant and uniform dissolution in water.

The inventors of the present invention surmised that in order to enable solubilized collagen fibers to be used as cosmetics, it was necessary to ensure that the components that constitute the fibers and the amounts of those components were distributed uniformly within the fibers, that the entire solubilized collagen fibers exhibited flexibility, and that when the fibers were dissolved in water, the entirety of the solubilized collagen fibers undergoing dissolution needed to make contact with the water. It was considered that conventional solubilized collagen fibers failed to fully consider these requirements.

### PRIOR ART DOCUMENTS

Patent Document 1: JP 07-83759 B
Patent Document 2: JP 4,356,654 B
Patent Document 3: JP 4,000,855 B
Patent Document 4: JP 2005-306736 A, JP 4,401,226 B
Patent Document 5: JP 2006-342472 A, JP 4,628,191 B

### DISCLOSURE OF INVENTION

### Problems Invention Aims to Solve

An object of the present invention is to provide solubilized collagen fibers having a novel form that can be used for cosmetics, wherein the operation of the drying step during production of the fibers is improved, the solubilized collagen fiber bundle is crimped, fiber separation defects (where the fiber bundle undergoes adhesion) do not exist, agglutination does not occur, the water content and the amount of hydrophilic organic solvent that exist within the fibers are reduced, and the residual water content and the residual hydrophilic organic solvent are dispersed as uniformly as possible within the various portions of the fibers, so that compared with conventionally known fibrous solubilized collagen fibers, the solubilized collagen fibers can be dissolved instantly and uniformly at the time of use.

### Means for Solution of the Problems

As a result of intensive research aimed at achieving the above object, the inventors of the present invention discovered adopted the following thinking.

(1) Looking at the methods used for producing conventional solubilized collagen fibers, it was thought that localized variations occurred in the fibers because the water content and the isopropyl alcohol that represents one possible hydrophilic organic solvent were unable to be distributed uniformly within the solubilized collagen fiber bundle during the drying step for the fiber bundle.

It was thought that by controlling the amounts of water and hydrophilic organic solvent that exist within the solubilized collagen fiber bundle during the drying step, the water content and the amount of the hydrophilic organic solvent within the solubilized collagen fiber bundle could be set within prescribed ranges. Further, it was also thought that the residual water content and the remaining hydrophilic organic solvent could be distributed so as to exist uniformly throughout the entire solubilized collagen fiber bundle.

(2) It was discovered that by using the technique described below as a drying method capable of achieving the above uniform distribution, the water content and the amount of the hydrophilic organic solvent could be controlled, meaning a solubilized collagen fiber bundle and solubilized collagen fibers having the intended water content and the intended amount of the hydrophilic organic solvent were able to be obtained.

Specifically, the technique described below was adopted. Namely, when drying the solubilized collagen fiber bundle and the solubilized collagen fibers, nip rollers were installed at the supply portion where the solubilized collagen fiber bundle was introduced into the drying treatment device, a portion of the water content and the organic solvent contained within the solubilized collagen fiber bundle was separated from the solubilized collagen fiber bundle by passing the bundle between the nip rollers, the solubilized collagen fiber bundle was subsequently introduced into a drying tube, sterilized air that had been controlled to a temperature of 30° C. or lower and a humidity of RH 70% or lower was forcibly blown through the tube to form a moving bed of air, and this moving bed of air was used to move the solubilized collagen fiber bundle for use in cosmetics, which had been reduced in weight due to the removal of a portion of the water content and the organic solvent by the nip rollers, thus enabling the production of a solubilized collagen fiber bundle in which the organic solvent and the water content were distributed uniformly from the inside to the outside of the solubilized collagen fiber bundle, and along the length direction of the solubilized collagen fiber bundle.

Moreover, by subjecting the solubilized collagen fiber bundle obtained using the above method to fiber opening, fibrous state solubilized collagen fibers could be obtained.

(3) In conventional solubilized collagen fibers, it is recognized that crimping does not occur, fiber separation defects (adhesion and agglutination of the fiber bundle) tend to occur, and the drying device does not act uniformly on the solubilized collagen fiber bundle, meaning that portions exist in the solubilized collagen fiber bundle in which the water content and the hydrophilic organic solvent are distributed non-uniformly. These problems do not exist in the solubilized collagen fiber bundle of the present invention, which is a solubilized collagen fiber bundle in which the organic solvent and the water content are distributed uniformly. It is thought that by using a solubilized collagen fiber bundle in this type of favorable state, the fiber bundle can be dissolved instantly and uniformly in water. Further, a solubilized collagen fiber bundle was disposed in a pile inside a container, and water was then injected into the top of the container to dissolve the fiber bundle. However, when the solubilized collagen fiber bundle was disposed in a pile and water was then injected onto the top of the pile, supplying the same amount of water uniformly to the entire solubilized collagen fiber bundle was problematic. Furthermore, it was also confirmed that dissolving the solubilized collagen fibers instantly and uniformly was impossible. It is thought that the reason for these observations is that by piling up the solubilized collagen fibers inside the container in a non-uniform manner, the fibers were unable to make contact with the water in a uniform manner, meaning instant dissolution of the fibers was impossible.

(4) With the device described above, even if solubilized collagen fibers in which the organic solvent and the water content are distributed uniformly within the fibers are used as novel solubilized collagen fibers, the object described above can still not be achieved. In order to enable the solubilized collagen fibers to be dissolved instantly and uniformly in water, in addition to using solubilized collagen fibers in which the organic solvent and the water content are distributed uniformly within the fibers, it is also important to ensure that all of the solubilized collagen fibers are able to contact the water at the same time. It is possible to form the solubilized collagen fibers as a substantially spherical fiber ball. The inventors of the present invention discovered that, in those cases where a mechanical apparatus was used, a fiber ball could be formed by placing the fibers in a closed state inside a groove or the like, and then conducting a rotational motion with the lump of the solubilized collagen fibers in contact with the wall surfaces of the groove or the like. The form of the fiber ball is as follows.

(i) A solubilized collagen fiber ball, comprising a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), having a diameter of 3 to 25 mm and a weight of 3 to 20 mg, and produced by forming, into a spherical shape, a bundle of solubilized collagen fibers having an average fineness of 3 to 10 dtx, a length of 1 to 20 cm and a wavy shape, in which the composition is distributed uniformly along a length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through the entire interior of the fiber ball in a state that is entangled in some areas and separated in other areas (see claim 6). Initially, insoluble collagen is solubilized using an alkali or a protein degrading enzyme to produce solubilized collagen fibers, and these fibers are passed through a fiber ball production apparatus provided with a helical groove in a cylinder or a flat plate-shaped fiber ball production apparatus having a spiral groove formed on a circular plate (see claims 11 and 12) to produce the fiber ball of claim 6.

(ii) An example of a handmade fiber ball is described in claim 1.

In this case, the inventors discovered that a manual operation could be used to produce a product having the actions of a fiber ball. Solubilized collagen fibers can be obtained in which all of the fibers make contact with the water at the same time when water is added. A fiber ball containing solubilized collagen fibers is produced by performing a solubilization step using an alkali or a protein degrading enzyme, spinning the fibers, and then conducting a drying step.

The target product is as described below. Namely, a solubilized collagen fiber ball, comprising a fiber bundle with a weight of 3 to 20 mg obtained by cutting solubilized collagen fibers which comprise a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), have an average fineness of 3 to 10 dtx and a wavy shape, and in which the composition is distributed uniformly along the length direction of the fibers (claim 1).

This enables a manual operation to be used to produce a rounded shape similar to a fiber ball using solubilized collagen fibers in a dry state.

(5) A certain period elapses from when the fiber ball is shipped from the factory as a product and marketed to consumers, until the consumer then uses the product, and it is necessary to ensure that the fiber ball maintains a stable state during this period.

The ability to obtain a solubilized collagen aqueous solution by dissolving solubilized collagen fibers in water is as described above. In terms of enabling a stable state to be maintained, it is essential that the solubilized collagen fibers are stored in individual packaging containers that contain only the amount of fibers required for a single use. The inside of these packaging containers must be shielded from the effects of changes in the external environment. In particular, in order to prevent the fibers from being affected by moisture within the air, air must be prevented from entering the container. For these reasons, a container capable of housing a solubilized collagen fiber ball that is sufficient for a single use is ideal. On the other hand, when using the solubilized collagen fibers, an amount of water is required that matches the amount of solubilized collagen fibers being used. A container was invented that had sufficient volume to contain the solubilized collagen fibers, as well as containing the amount of water required for use.

(6) Aspects of the present invention developed on the basis of the above thinking are as follows.

A. A solubilized collagen fiber ball, comprising a fiber bundle with a weight of 3 to 20 mg obtained by cutting solubilized collagen fibers which comprise a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), have an average fineness of 3 to 10 dtx and a wavy shape, and in which the composition is distributed uniformly along the length direction of the fibers.
B. The solubilized collagen fiber ball according to A, wherein the buffer salt is selected from among sodium citrate, sodium lactate and sodium phosphate.
C. The solubilized collagen fiber ball according to A or B, wherein the solubilized collagen fibers are obtained by (i) a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting the pH of the solubilized collagen aqueous solution in the presence of a buffer to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle from (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.
D. The solubilized collagen fiber ball according to A or B, wherein the solubilized collagen fibers are obtained by (i) a step of decomposing a skin sample containing insoluble collagen fibers using a protein degrading enzyme (protease) and extracting a solubilized collagen aqueous solution having an isoionic point of 7 to 8, and a step of adding an alkali to the solubilized collagen aqueous solution to adjust the pH to a value of 9 to 10, using a carboxylic acid anhydride to succinylate the solubilized collagen and reduce the isoionic point to 5 or less, subsequently precipitating and separating the solubilized collagen, and then adding an alkali in the presence of a buffer to adjust the pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.
E. The solubilized collagen fiber ball according to C or D, wherein the step of performing fiber opening to produce the targeted solubilized collagen fibers is performed by conducting fiber opening to obtain a fibrous state, and then using a grasping device to collect the required amount of fibers.
F. A solubilized collagen fiber ball, comprising a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), having a diameter of 3 to 25 mm and a weight of 3 to 20 mg, and produced by forming, into a spherical shape, a bundle of solubilized collagen fibers having an average fineness of 3 to 10 dtx, a length of 1 to 20 cm and a wavy shape, in which the composition is distributed uniformly along the length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through the entire interior of the fiber ball in a state that is entangled in some areas and separated in other areas.
G. The solubilized collagen fiber ball according to F, wherein the buffer salt is selected from among sodium citrate, sodium lactate and sodium phosphate.
H. The solubilized collagen fiber ball according to F or G, wherein the solubilized collagen fibers are obtained by (i) a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting the pH of the solubilized collagen aqueous solution in the presence of a buffer to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle obtained in (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.
I. The solubilized collagen fiber ball according to F or G, wherein the solubilized collagen fibers are obtained by (i) a step of decomposing a skin sample containing insoluble collagen fibers using a protein degrading enzyme (protease) and extracting a solubilized collagen aqueous solution having an isoionic point of 7 to 8, and a step of adding an alkali to the solubilized collagen aqueous solution to adjust the pH to a value of 9 to 10, using a carboxylic acid anhydride to succinylate the solubilized collagen and reduce the isoionic point to 5 or less, subsequently precipitating and separating the solubilized collagen, and then adding an alkali in the presence of a buffer to adjust the pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.
J. The solubilized collagen fiber ball according to H or I, wherein the step of performing fiber opening to produce the targeted solubilized collagen fibers is performed by conducting fiber opening to obtain a fibrous state, and then using a grasping device to collect the required amount of fibers.
K. The solubilized collagen fiber ball according to H or I, wherein the fiber ball is formed by supplying the solubilized collagen fibers to the end of a helical groove having a semicircular cross-section provided around a cylindrical outer surface of a cylinder, which is in a rotating state and is provided with a fixed cylindrical cover with a prescribed clearance between the cylinder and the cover, imparting a rotation to the solubilized collagen fibers sandwiched between the rotating helical groove and the fixed cover, moving the solubilized collagen fibers through the helical groove from one end of the cylinder to the other end of the cylinder, and using the rotational force imparted between the groove and the cover during movement through the groove to form the fiber ball.

The aforementioned solubilized collagen fiber bundle is converted to an expanded state, and moves along the helical groove while undergoing a rolling motion sandwiched between the cylindrical inner surface and the helical groove that revolves around this cylindrical inner surface.

The total length of the groove is 700 to 2,000 cm, the rate of revolution is 50 to 150 revolutions per minute, the clearance between the groove and the cover is 0.3 to 5 mm, and the rate of movement is determined by appropriate measurement. The diameter of the groove is set to a value either the same as, or slightly narrower than, the diameter of the fiber. The groove width may be uniform, or may be broader at the inlet and gradually narrow thereafter.

L. The solubilized collagen fiber ball according to H or I, wherein the fiber ball is formed using a flat plate-shaped fiber ball production apparatus, which comprises a circular plate that is rotated by a drive device and has a spiral groove with a semicircular cross-section formed in an upper surface that extends from the periphery of the plate toward the center, and a cover that is provided on top of the circular plate with a prescribed clearance therebetween, by supplying solubilized collagen fibers to a solubilized collagen fiber supply port provided at the periphery of the circular plate, imparting rotation to the solubilized collagen fibers sandwiched between the groove and the cover, moving the solubilized collagen fibers through the groove toward the center, and discharging a formed fiber ball from a solubilized collagen fiber outlet provided in the center of the circular plate

The aforementioned solubilized collagen fiber bundle is converted to an expanded state, is inserted in the space between the spiral groove provided in the circular plate and the cover, and moves along the spiral groove while undergoing a rolling motion caused by rotation of the circular plate.

The total length of the groove is 700 to 2,000 cm, the rate of revolution is 50 to 150 revolutions per minute, the clearance between the groove and the cover is 0.3 to 5 mm, and the rate of movement is determined by appropriate measurement. The diameter of the groove is set to a value either the same as, or slightly narrower than, the diameter of the fiber. The groove width may be uniform, or may be broader at the inlet and gradually narrow thereafter.

M. A container, wherein a container portion in which the solubilized collagen fiber ball defined in any one of A, B, F and G is disposed and a container portion for holding the water in which the solubilized collagen fiber ball is dissolved are integrated within a single container, the container portion in which the solubilized collagen fiber ball is disposed has a structure in which the side walls of the container narrow from the top toward the bottom, a horizontal flange is formed at the top of the container, and the top of the flange is sealed with a cover sheet.

### Effects of the Invention

According to the present invention, a novel solubilized collagen fiber bundle and a novel fiber ball with collagen fibers that is formed from solubilized collagen fibers can be obtained. As a result, when the solubilized collagen fiber bundle and the solubilized collagen fiber ball are used as cosmetics, they can be used as a solubilized collagen which, compared with fibrous state solubilized collagen fibers prepared using conventional methods, dissolves instantly and uniformly.

The solubilized collagen fiber ball comprises a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), has a diameter of 3 to 25 mm and a weight of 3 to 20 mg, and is produced by forming, into a spherical shape, a bundle of solubilized collagen fibers having an average fineness of 3 to 10 dtx, a length of 1 to 20 cm and a wavy shape, in which the composition is distributed uniformly along the length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through the entire interior of the fiber ball in a state that is entangled in some areas and separated in other areas.

### BRIEF DESCRIPTION OF THE DRAWINGS

FIG. 1 is a schematic structural diagram illustrating one example of an apparatus for producing solubilized collagen fibers according to the present invention.

FIG. 2 is a diagram illustrating conventional drying devices.

FIG. 3 is a diagram illustrating a drying apparatus of the present invention.

FIG. 4 is a diagram illustrating a container for storing and keeping a solubilized collagen fiber ball or a solubilized collagen fiber bundle of the present invention.

FIG. 5 is a diagram illustrating a container for storing and keeping a solubilized collagen fiber ball or a solubilized collagen fiber bundle of the present invention.

### EMBODIMENTS OF THE INVENTION

The target materials of the present invention are a novel solubilized collagen fiber bundle, and a novel fiber ball with collagen fibers that is formed from solubilized collagen fibers.

The raw material collagen is defined as a protein or glycoprotein having at least a partial helical structure (collagen helix). Collagen occurs as a triple helix composed of three polypeptide chains, wherein each polypeptide chain, which has a molecular weight of approximately 100,000, has a glycine residue every third position, and other frequently occurring amino acid residues include proline residues and hydroxyproline residues. Collagen is a protein that exists within all multicellular organisms, and can be extracted in large amounts from the tissues, and particularly the skin and bones, of invertebrates and vertebrates.

The raw material for the solubilized collagen fibers of the present invention is the collagen described above. This type of collagen is insoluble collagen, is contained within the skin tissue and other organs of animals such as cows, pigs, birds and fish, and is obtained from tissues that contain such insoluble collagen.

The inventors of the present invention initially started their research into collagen production with the aim of effectively utilizing the split leather generated as a by-product during the production of leather. This split leather can be used as the raw material.

Subsequently, as leather production shifted to tanning production methods (methods that use wet blue and wet white for leather production), split leather was no longer generated.

The tissues containing insoluble collagen described above are now used as the raw material for the purpose of producing collagen.

Examples of raw materials that can be used for solubilized collagen fibers for cosmetics include mammal hides, and tissue derived from aquatic organisms such as fish skin and fish scales. By selecting the raw material used for obtaining the collagen, a difference is observed in the denaturation temperature of the obtained collagen. When the raw material is in a dried state, there are no particular differences in the handling methods used, regardless of the raw material from which the solubilized collagen is derived. Currently, because of issues relating to BSE, the use of bovine-derived insoluble collagen tissue is undesirable, and the use of either porcine-derived collagen or collagen derived from an aquatic organism such as fish is preferable.

Recently, the production of collagen from synthetic peptides is garnering much attention as a potential material that suffers no danger of B SE infection. A novel polypeptide of the present invention comprises a peptide unit having an amino acid sequence represented by formula (1) shown below, and a peptide unit having an amino acid sequence represented by formula (2) shown below.

-Pro-X-Gly- (1)

—Y—Z-Gly- (2)

In these formulas, X and Z may be the same or different, and each represents Pro or Hyp, and Y represents an amino acid residue having a carboxyl group (such as Asp, Glu or Gla).

The ratio (molar ratio) between the above peptide unit (1) and the peptide unit (2) is within a range from approximately (1)/(2)=99/1 to 1/99. The polypeptide may also support apatites (see JP 4,303,137 B).

Examples of the collagen used as the raw material for the solubilized collagen fibers of the present invention include pig skin that has undergone alkali solubilization, and pig skin that has undergone enzyme solubilization and succinylation to adjust the isoionic point to an acidic value. However, the list of raw materials that can be used is not limited to the above materials, and materials produced by subjecting fish skin or fish scales to a solubilization treatment can also be used. The collagen material for use in the present invention includes materials which have an isoionic point that is sufficiently removed from the neutral region that is ideal for cosmetics, either toward the low side (acidic) or toward the high side (alkaline), and which are highly soluble in water in the neutral region but solidify within organic solvents. Provided these conditions are satisfied, synthetic collagen can also be used.

The properties of the solubilized collagen fibers of the present invention are as described below.

Namely, a solubilized collagen fiber ball, comprising a fiber bundle with a weight of 3 to 20 mg obtained by cutting solubilized collagen fibers which comprise a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), have an average fineness of 3 to 10 dtx and a wavy shape, and in which the composition is distributed uniformly along the length direction of the fibers.

The buffer salt is selected from among sodium citrate, sodium lactate and sodium phosphate.

The solubilized collagen fibers composed of a fiber bundle described above differ from the solubilized collagen fiber ball according to the present invention described below. The aforementioned solubilized collagen fibers composed of a fiber bundle according to the present invention refer to a fiber ball state that is prepared without including a step for forming the fiber ball using a mechanical apparatus. The present invention is not limited to ideal ball shapes such as the solubilized collagen fiber ball of the present invention produced using a mechanical apparatus in a step described below, and also includes products having distorted shapes, and products that include many portions in which the shape of the fiber bundle is retained. These products can also be used satisfactorily as cosmetic materials.

The properties of the solubilized collagen fiber ball are as follows.

Namely, the solubilized collagen fiber ball comprises a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), has a diameter of 3 to 25 mm and a weight of 3 to 20 mg, and is produced by forming, into a spherical shape, a bundle of solubilized collagen fibers having an average fineness of 3 to 10 dtx, a length of 1 to 20 cm and a wavy shape, in which the composition is distributed uniformly along the length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through the entire interior of the fiber ball in a state that is entangled in some areas and separated in other areas.

Further, the buffer salt is selected from among sodium citrate, sodium lactate and sodium phosphate.

Formation of the above solubilized collagen fiber ball is conducted using solubilized collagen fibers comprising a composition having a solubilized collagen content of 66 to 87 wt %, a buffer salt content of 2 to 6 wt %, a water content of 10 to 22 wt % and a residual hydrophilic organic solvent content of a trace amount to 6 wt % (totaling 100 wt %), wherein the fibers have an average fineness of 3 to 10 dtx and a wavy shape, and the composition is distributed uniformly along the length direction of the fibers.

The solubilized collagen fiber ball is formed from these solubilized collagen fibers.

The weight of the solubilized collagen fiber ball must be controlled within a range from 5 to 20 mg, and preferably from 10 to 15 mg. The concentration of the solubilized collagen used is from 0.5 wt % to 1.5 wt %.

If the amount used in a single application is less than 3 mg, then achieving an adequate moisturizing effect and a satisfactory sensation becomes difficult, whereas if the amount exceeds 20 mg, then the amount of product requiring application becomes too great, causing a film-like feeling or a tightening sensation. The ideal amount is affected by the properties of the skin of the user and the surrounding humidity and temperature, but an amount within a range from 10 to 15 mg generally yields no complaints from the majority of users.

The solubilized collagen fiber ball is formed with a spherical shape having a diameter within a range from 3 to 25 mm, and preferably from 5 to 20 mm. If the size is less than 5 mm, then space between fibers becomes overly dense. Further, uniform dissolution of the fiber ball in water becomes impossible, and the fiber ball is prone to forming a lump. If the size exceeds 20 mm, then the spacing between fibers becomes very sparse and the degree of fiber entanglement is inadequate, meaning the shape of the fiber ball cannot be maintained, making handling difficult.

Fineness Range

In order to enable formation of the solubilized collagen fiber ball, fibers having a fineness of 3 dtx to 10 dtx are used as the solubilized collagen fibers. The fineness was measured using a fineness meter (Deniel Computer DC-11A, manufactured by Search Co., Ltd.), by measuring 20 fibers from each sample in an environment at 20° C. and 65% RH, and then calculating the average fineness value.

Further, in a simplified method, the fiber diameter was measured using a microscope, and the fineness then calculated from the correlation formula between the fineness and the fiber diameter. If solubilized collagen fibers having a fineness of 3 dtx or less are used, then the fiber strength of the solubilized collagen fibers is too weak, and considerable fiber breakage occurs during fiber opening, increasing the occurrence of lint-like fibers having a fiber length of only several mm. If a solubilized collagen fiber ball is formed using such solubilized collagen fibers, then the problems outlined above mean that a satisfactory solubilized collagen fiber ball cannot be obtained.

If fibers having a fineness exceeding 10 dtx are used as the solubilized collagen fibers, then a longer than intended dissolution time is required when producing the solubilized collagen fibers. Further, if a solubilized collagen fiber ball is formed using solubilized collagen fibers having a fineness exceeding 10 dtx, then the feel of the product becomes overly hard, which is undesirable. If an attempt is made to prepare this type of product, then agglutination tends to occur during drying when producing the solubilized collagen fibers.

Fiber Length Range

The fiber length is within a range from 1 to 20 cm, and preferably from 3 to 15 cm (the numerical value associated with current fiber opening conditions). Fibers having a variety of different lengths within this range may be mixed. If the length is 1 cm or less, then insufficient entanglement means that lint-like fibers are more likely to fall out of the product, whereas if the length is 20 cm or greater, then during formation of the solubilized collagen fiber ball, the operation of arranging the fibers becomes more complicated.

In order to convert the solubilized collagen fibers to fiber balls with solubilized collagen fibers, an apparatus is used that represents an improved fiber ball production apparatus, and the operating conditions for the apparatus are also modified.

Methods and apparatus for forming a material in a fibrous state into fiber balls are already known (such as JP 2001-295170 A, JP 3,601,004 B and JP 10-266051 A). These production methods and production apparatus relate to the formation of fibers including synthetic fibers such as polyester and polypropylene, animal fibers such as wool, or metal fibers of stainless steel or the like into a spherical shape. The thus produced fiber balls are used as filter materials for water tanks or septic tanks or the like, or as cushioning materials in healthy pillows and the like, and these products require fiber balls in which the surface exists in a dense state, and the interior is composed mainly of space. Besides the fiber balls described above, fiber balls prepared by forming absorbent cotton into a spherical shape are also known as medical items (such as JP 3,557,587 B). The absorbent cotton is rolled on a planar surface to form a fiber ball.

The solubilized collagen fibers used in the present invention contain both water and isopropyl alcohol (the hydrophilic organic solvent), and even if heat is applied to dry the fibers, in order to avoid denaturation, the temperature must be controlled at 30° C. or lower.

Solubilized collagen fibers have less rigidity and elasticity than the fibers described above.

For these reasons, if the apparatus described above are used, then production of the fiber balls with solubilized collagen fibers must be performed under conditions that are appropriate for the solubilized collagen fibers used in the present invention.

When producing a fiber ball using the apparatus described above, a rotational force is imparted to the material being treated. As this rotational force is imparted, pressure is applied, and the number of revolutions is set to not less than a prescribed value. When a fiber ball of solubilized collagen fibers is formed using this type of fiber ball production apparatus, the pressure is lowered and the number of revolutions is also lowered. The temperature applied for forming the fiber ball is lowered to 30° C. or lower. Further, the conditions are set so that the frictional force applied to the solubilized collagen fibers inside the apparatus does not become too high, causing an unstable state.

The cross-sectional shape of the groove in the apparatus is semicircular. The width of the groove is set to a value either the same as, or slightly narrower than, the diameter of a typically designed solubilized collagen fiber ball. Following production of a solubilized collagen fiber ball, the shape of the solubilized collagen fiber ball tends to expand, and therefore making the width of the groove slightly narrow than the diameter of the fiber ball is desirable. For example, when fiber balls having a diameter of 10 mm are to be produced, the groove width may be set to 8 mm. When fiber balls having a diameter of 18 mm are to be produced, the groove width is set to 15 mm.

The width of the groove may be a uniform width along the entire length of the groove, but if the width is made broader at the fiber ball inlet, and then narrows gradually toward the outlet, then failures during introduction of the fiber ball into the groove can be reduced. If the groove width inlet is the same as, or slightly narrower than, the diameter of the fiber ball, then accidental introduction of the fiber at a portion where the groove has not been cut tends to occur frequently. Even if the groove width at the inlet is widened, provided that the groove width at the outlet is set to a value either the same as, or slightly narrower than, the diameter of the fiber ball, fiber balls of the intended size can be obtained, although in order to ensure that the fibers exist in a state of satisfactory entanglement, the length of the groove having the same width as the outlet is preferably at least 1,000 cm.

The clearance between the groove and the cover is set to 0.5 to 5 mm. If the clearance exceeds 5 mm, then the fiber balls can extend beyond the groove and fall back into the adjacent groove, causing the rate of progression of the fiber through the apparatus to slow, and causing fiber balls to aggregate together. The length of the groove is set to 700 to 2,000 cm. If the length of the groove is less than 700 cm, then either a spherical shape is not formed, or even if a spherical shape is formed, the degree of fiber entanglement is unsatisfactory, meaning the formed shape is prone to collapse. A groove length of 2,000 cm is sufficient to ensure a good state of entanglement, and there is no need to extend the groove length beyond 2,000 cm. The apparatus comprises a fixed cylindrical member having a cylindrical inner surface formed as a high-friction surface, and a rotating member having a helical groove formed in the surface of a high-friction surface that rotates around the inner surface of the fixed cylindrical member, and fibers in the form of an aggregated lump are subjected to a rolling motion sandwiched between the cylindrical inner surface and the helical groove, thus forming a spherical shape as they move. A cloth surface or a roughened rubber sheet or the like is used as the high-friction surface.

Besides improving the above fiber ball production apparatus, the inventors of the present invention also investigated the effects of the frictional resistance between the fiber ball and the groove and the cover, and the clearance between the groove and the cover, and developed and tested a novel cylindrical fiber ball production apparatus and a flat plate-shaped fiber ball production apparatus that are ideal for forming solubilized collagen fibers into fiber balls. The technique of rolling a lump of the solubilized collagen fibers between a groove and a cover to form the fiber balls is the same as the aforementioned fiber ball production apparatus, and conditions such as the shape and the width of the groove are the same as those described above for the improved fiber ball apparatus.

In the cylindrical fiber ball production apparatus, the solubilized collagen fibers are supplied to the end of a helical groove having a semicircular cross-section provided around the outer surface of a cylinder. The cylinder is provided with a cylindrical cover with a prescribed clearance between the cylinder and the cover. The cylinder having the helical groove provided therein is provided with a drive device, and during operation, the cylinder itself is rotated continuously by this drive device. The solubilized collagen fibers are rotated and rolled by sandwiching the fibers between the rotating helical groove and the stationary cover, and move through the helical groove from one end of the cylinder to the other. During this movement, the fibers are rolled between the groove and the cover, thus forming fiber balls with solubilized collagen fibers.

The total length of the groove is 700 to 2,000 cm, the rate of revolution is 50 to 150 revolutions per minute, the clearance between the groove and the cover is 0.3 to 5 mm, and the rate of movement is determined by appropriate measurement.

The diameter of the groove is set to a value either the same as, or slightly narrower than, the diameter of the fiber. The groove width may be uniform, or may be broader at the inlet and gradually narrow thereafter.

Further, the flat plate-shaped fiber ball production apparatus comprises a circular plate which is provided with a spiral groove with a semicircular cross-section that extends from the periphery of the plate toward the center, and a cover that is provided on top of the circular plate with a prescribed clearance therebetween. The circular plate is provided with a drive device, and when the circular plate is rotated by the drive device, and the solubilized collagen fibers are inserted into the spiral groove from a solubilized collagen fiber supply port provided at the periphery of the circular plate, a rotational force is imparted to the solubilized collagen fibers sandwiched between the groove and the cover, moving the solubilized collagen fibers through the groove toward the center, and discharging the formed fiber balls from a solubilized collagen fiber outlet provided in the center of the circular plate.

The total length of the groove is 700 to 2,000 cm, the rate of revolution is 50 to 150 revolutions per minute, the clearance between the groove and the cover is 0.3 to 5 mm, and the rate of movement is determined by appropriate measurement. The diameter of the groove is set to a value either the same as, or slightly narrower than, the diameter of the fiber. The groove width may be uniform, or may be broader at the inlet and gradually narrow thereafter.

In terms of the surface and interior of the fiber balls with solubilized collagen fibers formed from the solubilized collagen fibers, the bulk density of the fiber balls is 4.0 to 8.0 mg/cm³, the fiber ball diameter is 3 to 25 mm, and inside the fiber ball, each solubilized collagen fiber is distributed through the entire interior of the fiber ball in a state that is entangled in some areas and separated in other areas.

The shape and size of the solubilized collagen fiber ball is as follows.

The weight of the solubilized collagen fiber ball is determined with due consideration of the amount used of the solubilized collagen fiber solution obtained by dissolving the fiber ball in water. The concentration of the solubilized collagen and the amount used of the solubilized collagen can be determined appropriately in accordance with factors such as the area of the portion to which the user intends to apply the solution, and the concentration of the solution used for the application. These amounts may change depending on the state of the skin of the user. A decision to either increase the amount of the fiber ball used in a single application, or decrease the amount and retain the remainder for another use, can be made with due consideration of the state of the user. Further, the fact that the fiber balls can be used in this type of manner is another advantage of the fiber balls with solubilized collagen fibers.

The amount used of the solubilized collagen fiber ball in a single use assumes a weight for each fiber ball of 5 to 20 mg. It is assumed that the concentration of the solubilized collagen in a single use is within a range from 0.5 to 1.5 wt %. The volume of water used must therefore be from 0.5 to 1.5 ml.

In terms of the container used for the solubilized collagen fiber ball, consideration of the ease of operation when dissolving the fiber ball inside the container using a finger, and the requirement to store and keep the fiber ball until it used means a container having a prescribed volume is used. The volume is assumed to be 5 to 10 ml. In some cases, the volume of the container may be insufficient for the amount of water used. In such cases, the user may adjust the amount of water used as appropriate.

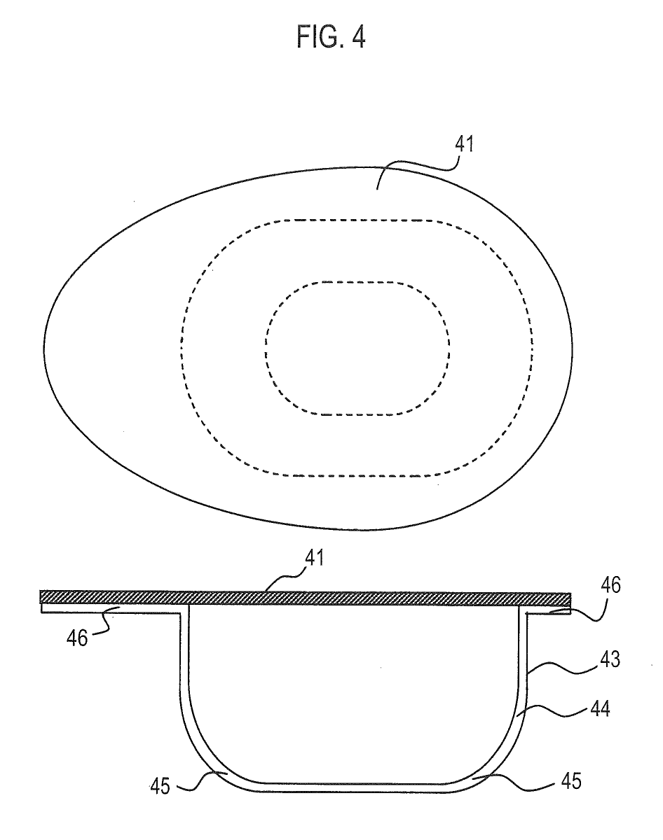

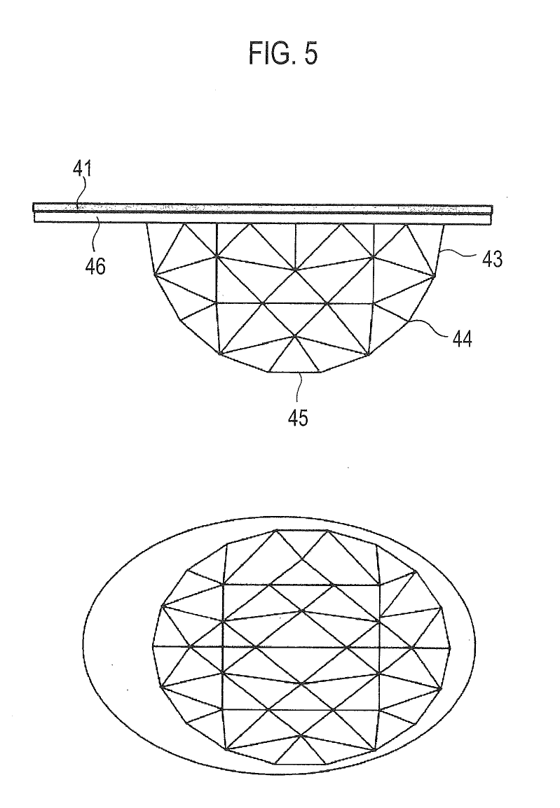

Examples of packing containers for filling with either a collagen fiber bundle cosmetic material or a fiber ball-shaped collagen cosmetic material are illustrated in FIG. 4 and FIG. 5.

The upper portion of FIG. 4 illustrates the upper surface of the top cover. The lower portion of FIG. 4 illustrates a longitudinal sectional view.

A container portion 43 in which the solubilized collagen fiber ball is disposed, and a container portion 44 for holding the water in which the solubilized collagen fiber ball is dissolved are integrated within a single container, and the container portion in which the solubilized collagen fiber ball is disposed has a structure 45 in which the side walls of the container narrow from the top toward the bottom (meaning the surface area of the container cross-section gradually decreases). In the example illustrated in FIG. 4, the side walls have a gently curved surface. A horizontal flange 46 is formed at the top of the container, and the top of the flange is sealed with a top cover sheet 41.

Another example of a packing container for filling with a collagen fiber bundle cosmetic material or a fiber ball-shaped collagen cosmetic material is illustrated in FIG. 5.

The upper portion of FIG. 5 illustrates the upper surface of the top cover. The lower portion of FIG. 5 illustrates a longitudinal sectional view.

A container portion 43 in which the solubilized collagen fiber ball is disposed, and a container portion 44 for holding the water in which the solubilized collagen fiber ball is dissolved are integrated within a single container, and the container portion in which the solubilized collagen fiber ball is disposed has a structure 45 in which the side walls of the container narrow from the top toward the bottom (meaning the surface area of the container cross-section gradually decreases). In the example illustrated in FIG. 5, the side walls constitute a polyhedron composed of a combination of small triangular (planar) surfaces. A horizontal flange 46 is formed at the top of the container, and the top of the flange is sealed with a top cover sheet 41.

The material of the container main body is a material that blocks light and can prevent the transmission of oxygen and moisture, and an example of a material that can be used is PBP (a material in which an ethylene-vinyl alcohol copolymer resin (EVOH) is sandwiched between polypropylene chains). This material is used because collagen is prone to crosslinking in the presence of light (and particularly ultraviolet light), moisture, and aldehydes (which may be generated if plywood is used in the storage location). By using a material that blocks light and is impermeable to oxygen and moisture, any deterioration in the solubility caused by crosslinking of the collagen during storage can be prevented.

In terms of the external shape of the bottom of the container, the bottom portion 42 is a rounded shape. This prevents the solubilized collagen aqueous solution obtained by dissolution in water from adhering to corner portions and becoming difficult to remove from the container. FIG. 4 illustrates an example in which the side walls are formed as a gently curved surface so as to avoid the formation of corner portions. In the example illustrated in FIG. 5, the container is a polyhedron composed of a combination of small triangular (planar) surfaces, and although corner portions are formed at the points where the planar surfaces meet, provided the angle of the corner portions is sufficiently large, adhesion of the solubilized collagen aqueous solution within these corner portions can be prevented.

In a similar manner to the container main body, the material for the cover also uses a material that blocks light and is impermeable to oxygen and moisture. As a result, external air and light are blocked from entering the portion containing the fiber ball, meaning the fiber ball can be stored in a favorable state.

One example of the shape of the container is a container having a length of 30 mm, a width of 25 mm and a depth of 20 mm. The material for the cover employed a structure in which a PET layer was laminated on top of aluminum. The cover can be sealed by fusion to the container main body.

The functions of the container are as follows.

Because the solubilized collagen fiber ball is composed of fine collagen fibers, it dissolves instantly in water and can be used immediately. Because the amount of fiber for a single use is packaged individually, it is easy to use. The inside of the container is of a shape and size that enables the solution to be prepared easily using a finger. Because the fiber ball is a dried product, denaturation and decomposition do not occur, and there is no need to use low-temperature transport or to add preservatives. The container is formed from a material that blocks light and is impermeable to oxygen and moisture, and can therefore be used effectively to prevent any deterioration in the solubility of the collagen fibers caused by crosslinking during storage.

It is assumed that the concentration of the solubilized collagen aqueous solution obtained by dissolving the solubilized collagen fiber ball in water will be within a range from 0.5 to 1.5% (by weight). When a 10 mg solubilized collagen fiber ball is used, the amount of water used is approximately 0.7 to 2.0 ml.

The aqueous liquid used in the fiber ball-shaped collagen cosmetic material is prepared so that the pH of the collagen solution is removed from the isoionic point of collagen, and usually employs a solvent medium composed mainly of water.

In those cases where pure water is used as the solvent medium, the solubility of the fiber ball-shaped collagen cosmetic decreases due to the effect of the buffering action by the collagen of the solubilized collagen fibers. In order to prevent this type of decrease in the solubility, an electrolyte is incorporated within the fiber ball-shaped collagen. By adding a small amount of an electrolyte such as an acid, base, neutral salt or buffer salt, the fiber ball-shaped collagen is able to be dissolved satisfactorily within aqueous liquids. In particular, if a buffer salt such as sodium citrate, sodium lactate or sodium phosphate (namely, a salt of a weak acid and a weak base) that stabilizes the pH in a range from slightly acidic to neutral is added to the aqueous liquid to adjust the pH of the aqueous liquid to a value of approximately 5.5 to 9.0, then the dissolution of the solubilized collagen fibers for cosmetics can be stabilized, and solubilized collagen fibers for cosmetics having an average fineness of approximately 10 dtx or less can be dissolved within a period of 30 seconds. If an excessive amount of the salt is present, then a salting-out effect makes it difficult to dissolve the collagen in aqueous liquids. The electrolyte may be incorporated within an aqueous solution.

In regard to this point, because the buffer salt or neutral salt or the like incorporated within the solubilized collagen solution does not migrate totally into the organic solvent during spinning, residual electrolyte exists within the fiber ball-shaped collagen cosmetic. In this case, the solubilized collagen may also be used in this state, with no further modification.

Examples of aqueous solutions that can be used include commercially available toilet waters and cosmetic liquids. Because of their favorable properties, the novel solubilized collagen fiber bundle for cosmetics, and the novel solubilized collagen fiber ball formed from solubilized collagen fibers according to the present invention dissolve rapidly in commercially available toilet waters and cosmetic liquids. Accordingly, the user may select a toilet water or cosmetic liquid depending on individual preference, and then combine this selected water or liquid with the solubilized collagen fibers or fibrous material for cosmetic use, thus preparing a solubilized collagen solution for cosmetic use. Thus, a solubilized collagen cosmetic material that satisfies the needs of the user can be provided to the user in a fresh state whenever it may be required. Depending on the state of the skin of the user, a cosmetic that is suitable for that skin state can be prepared. The type of cold-temperature storage required for conventional solubilized collagen cosmetic materials is unnecessary, and the time required for preparing the cosmetic material is short, meaning there is no time restriction associated with use of the product, and the product may simply be used in accordance with the needs of the user.

Following dissolution, the collagen cosmetic material is prone to denaturation in a similar manner to that observed for typical collagen cosmetics in the form of aqueous solutions. However, the aforementioned treatment in which an alcohol was used as the organic solvent during the preparation of the solubilized collagen fibers has a sterilization effect on the collagen, and therefore the resulting solubilized collagen fibers, which are obtained by drying with sterilized air, are not contaminated with unwanted bacteria. Moreover, compared with collagen in a solution state, solubilized collagen in a dried state is significantly more resistant to the proliferation of bacteria or mold or the like, meaning the level of treatment required to preserve the product during transport can be reduced. Cosmetic materials that contain almost no components other than the collagen, including components such as preservatives, can be used.

Any of the various components typically added to solubilized collagen fibers for use in cosmetics may be added to the aqueous liquid, provided this addition does not impair the dissolution of the solubilized collagen in an aqueous solution. Examples of these components include moisturizing agents such as butanediol, pentanediol, glycerol, hyaluronic acid and urea, preservatives such as methyl p-hydroxybenzoate and phenoxyethanol, plant extracts such as aloe extract, alcohol-based solvents such as ethanol, ultraviolet absorbers, vitamins, anti-inflammatory agents, oils and fats such as olive oil, fatty acids, and any of the various functional components that have a specific cosmetic function.

By setting the combination ratio between the collagen fibers and the aqueous liquid so that the collagen content within the obtained cosmetic material is approximately 0.01 to 10 mass %, and particularly approximately 0.1 to 3 mass %, a uniformly dissolved cosmetic material can be obtained rapidly.

Similarly, in the case of aqueous liquids for use as cosmetics, because separation is performed from collagen having a high nutritional value, the amount of added preservatives can be reduced, and the level of preservation treatment required can be reduced. Further, aqueous liquids can be sterilized more easily than collagen, and therefore by sterilizing the aqueous liquid and using aseptic packaging, the addition of preservatives becomes unnecessary.

Methods for producing the solubilized collagen fibers used for obtaining the fiber ball-shaped collagen are described below.

These methods include (1) a method for performing production under alkaline conditions, and (2) a method for performing production using an enzyme. The features of each method are described below.

(1) All of the steps in the method used for decomposing the insoluble collagen fibers that function as the raw material under alkaline conditions to obtain the solubilized collagen fibers of the present invention are described below.

The production apparatus of the present invention and all of the steps described in the present invention are conducted in an environment that is maintained in a sterilized state.

(i) A step of decomposing insoluble collagen fibers under alkaline conditions and extracting a solubilized collagen aqueous solution, and a step of performing pH modification to produce a solubilized collagen aqueous solution that functions as a solubilized collagen fiber raw material.

Namely, a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting the pH of the solubilized collagen aqueous solution in the presence of a buffer to a value within a range from 6.0 to 7.5 which is greater than the isoionic point, thus preparing a solubilized collagen aqueous solution that functions as a solubilized collagen fiber raw material.

These steps are described below in further detail.

A method of treating insoluble collagen fibers with a material obtained by adding a small amount of an amine or an analog thereof to an aqueous solution containing both a caustic alkali and sodium sulfate (for example, see JP 46-15033 B, hereafter this method is referred to as the “alkali treatment method”) is described below.

The dermic layer is extracted from the raw hide containing the insoluble collagen that functions as the raw material, and a wet grinding mill is used to convert the dermic layer to a paste form that undergoes reaction more readily.

In the alkali treatment method, a strong alkaline composition containing approximately 4 to 5% of sodium hydroxide, approximately 10 to 12% of sodium sulfate, and approximately 1% of monomethylamine (wherein the above numbers represent weight concentrations within the solution) is used as the alkali treatment agent.

The sodium hydroxide within the strong alkaline composition hydrolyzes the peptides of the collagen crosslinked portions (telopeptides), thereby accelerating the solubilization. The sodium sulfate is used for preventing swelling of the collagen by the alkali. If the monomethylamine is not used, then the solubilization tends to be unsatisfactory, and a hard viscous solution (containing a large amount of multimers) is obtained.

During the solubilization treatment, it is necessary to ensure that denaturation of the collagen and precipitation of the sodium sulfate do not occur, and therefore the temperature of the solubilization treatment tank is maintained within a range from 22° C. to 27° C.

The above treatment yields a product containing a solubilized collagen. By subjecting this product to a neutralization treatment, the solubilized collagen is retained in the form of neutralized and desalted skin, and this neutralized and desalted skin can be separated by a solid-liquid separation using a net-like device that allows the passage of water, such as a sieve. Alternatively, the neutralized and desalted skin can be separated by a centrifugal separation method using a low centrifugal force.

As a result of the solid-liquid separation, a neutral solid containing the solubilized collagen can be extracted. This neutral solid is then washed and desalted to obtain the targeted neutralized and desalted solid of solubilized collagen.

The neutralized and desalted solid of solubilized collagen obtained from the above treatment may then be stirred within an acidic solution such as lactic acid to obtain a solubilized collagen aqueous solution.

In the alkali treatment, the isoionic point of the obtained solubilized collagen is from 4.8 to 5.0. This is because the asparagine residues and glutamine residues in the collagen undergo a deamidation (releasing free ammonia) in the presence of the alkali, and are converted to aspartic acid residues and glutamic acid residues respectively.

Cosmetic items are preferably within a range from slightly acidic to neutral, and therefore during preparation of the solubilized collagen fiber raw material for use in cosmetics, no dramatic change is required in the isoionic point of the solubilized collagen. The collagen concentration of the collagen fiber raw material aqueous solution is generally within a range from approximately 3 wt % to 6 wt %.

The pH of the solubilized collagen aqueous solution is adjusted in the presence of a buffer to a value within a range from 6.0 to 7.5 which is greater than the isoionic point, thus preparing a solubilized collagen aqueous solution that functions as the solubilized collagen fiber raw material.

Following preparation of the solubilized collagen fibers, adjusting the pH of the solubilized collagen aqueous solution that functions as the solubilized collagen fiber raw material is effective in obtaining a solubilized collagen aqueous solution that can be used as a cosmetic.

The reasons for this observation are as follows.

Collagen is an amphoteric electrolyte, and has a property wherein the electric charge varies depending on the pH. The pH at which the positive and negative charges are in equilibrium, resulting in an apparent charge of zero, is the isoionic point. At this pH, the solubility of collagen deteriorates and aggregation tends to occur. Accordingly, in order to improve the solubility in the neutral region that is desirable for cosmetics, it is important that the isoionic point is somewhat removed from the neutral region. In the present invention, performing the alkali treatment alters the isoionic point to a value of 4.5 to 5.0. Alternatively, a method may be employed in which collagen having an isoionic point of approximately 7 to 8 obtained from a solubilization treatment that uses a protease enzyme is subjected to a chemical treatment such as succinylation to lower the isoionic point. The solubilization is performed to enable spinning of the obtained collagen. If the pH is held at the isoionic point, then dissolution is impossible, and therefore the solubilization must be performed on either the acidic side or the alkali side of the isoionic point. However, when solution preparation is performed on the acidic side of the isoionic point (for example, pH 3), then when an attempt is made to subsequently dissolve the obtained dried fibers in a neutral aqueous liquid (for example, pH 7) for use as a cosmetic, because the pH must pass through the isoionic point, aggregation may occur and dissolution takes a considerable length of time, meaning use as a cosmetic is problematic. On the other hand, when the solution is prepared on the alkali side of the isoionic point, and particularly at a pH within a range (pH 6.0 to 7.5) that is close to that at which final dissolution of the dried fibers occurs, the solution need not pass through the isoionic point, and the collagen remains in a readily dissociated state, meaning rapid dissolution can be achieved, and collagen fibers that are ideal for use as cosmetics can be obtained.

(ii) A step of subjecting the solubilized collagen aqueous solution that functions as the solubilized collagen fiber raw material to spinning and drawing to produce a solubilized collagen fiber bundle.

Namely, a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent.

Specifically, this step is conducted in the manner described below.

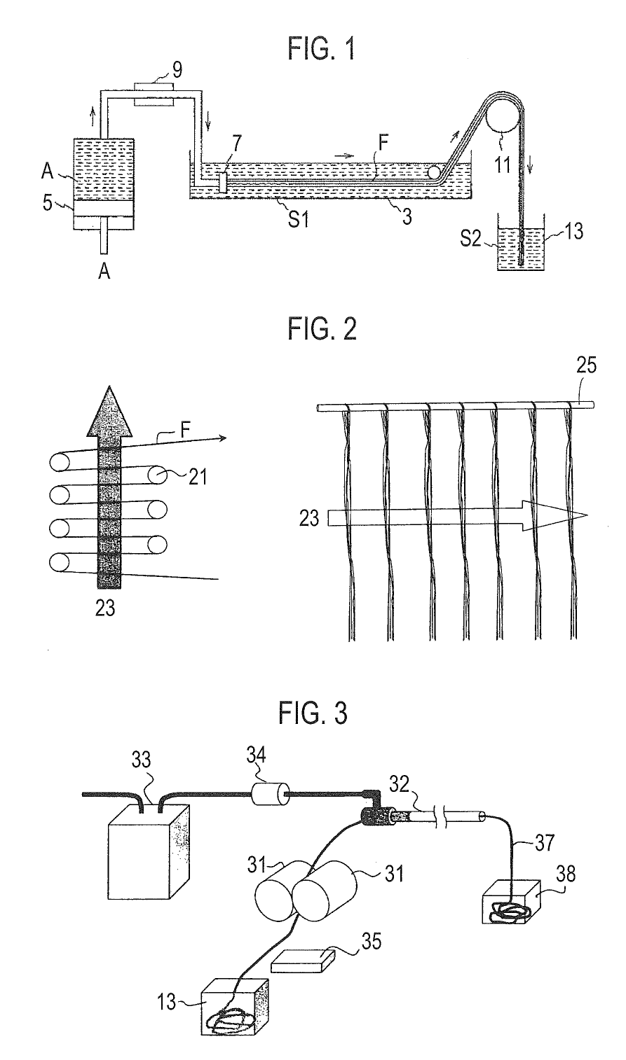

FIG. 1 is a diagram illustrating one example of a production apparatus for producing the type of solubilized collagen fibers described above.

This production apparatus 1 comprises a piston tank 5, which holds a solubilized collagen aqueous solution A and supplies the solubilized collagen aqueous solution A, a first solvent tank 3 containing an organic solvent S1 composed of isopropanol into which the supplied solubilized collagen is discharged via a nozzle 7 having a plurality of discharge holes, and in which the discharged collagen is spun and then drawn, enabling the extraction of solubilized collagen fibers containing water, a winding roller 11 which is wound at a predetermined winding rate and is used for drawing and extracting the fibers in the form of solubilized collagen fibers having a water content, and a second solvent tank 13 containing a hydrophilic organic solvent S2 into which the solubilized collagen fibers containing water that have been wound by the roller 11 are supplied.

Further, the supply of the solubilized collagen aqueous solution A from the piston tank 5 through the nozzle 7 is performed through the action of a gear pump 9. The winding roller 11 is used for winding the spun solubilized collagen fibers at a predetermined winding rate.

The piston tank 5 and the nozzle 7 are connected, via the gear pump 9, by a plastic conduit. In this example, the first solvent tank 3 has an elongated shape of a prescribed length, and the nozzle 7 is installed at one end of the first solvent tank 3 with the discharge holes directed in the horizontal direction, enabling the collagen aqueous solution discharged from the nozzle 7 to travel horizontally through the organic solvent S1 along the length of the first solvent tank 3 to the other end of the first solvent tank 3.

When the solubilized collagen aqueous solution is discharged into the organic solvent and solidified, the organic solvent used may be either a hydrophilic organic solvent or a hydrophobic organic solvent.

The collagen that solidifies within the organic solvent incorporates water, and this incorporated water must be diffused externally. In terms of facilitating the external diffusion of the water away from the fibers, a hydrophilic organic solvent is particularly suitable.

In order to enable efficient drying of the solidified fibers, the use of a solvent that enables the water to be evaporated from a state that incorporates water is desirable, and in this regard, the use of a hydrophilic organic solvent is preferred. Specific examples of solvents that can be used include alcohols such as methanol, ethanol, and isopropanol, and acetone. A mixed solvent containing a plurality of different solvents may also be used. From a practical perspective, an organic solvent containing a small amount of water can also be used, and in such cases, the water content within the solvent is typically not more than approximately 15 mass % and preferably 10 mass % or less. If the water content is too high, then the collagen cannot be solidified favorably.

In the production apparatus 1 illustrated in FIG. 1, when the piston of the piston tank 5 is pressurized using compressed air, and the gear pump 9 is activated, the solubilized collagen aqueous solution A is supplied from the piston tank 5 to the nozzle 7, and is then discharged into the organic solvent S1 inside the first solvent tank 3 from the plurality of circular discharge holes in the nozzle 7.

The solubilized collagen is discharged into the organic solvent from the plurality of circular discharge holes in the nozzle 7, solidification of the solubilized collagen proceeds from the external surface toward the interior, resulting in fiber formation, and by pushing the collagen out in a horizontal direction, the plurality of collagen fibers are spun into a bundle while also undergoing a drawing treatment. The formed bundle of the solubilized collagen fibers F is pulled out of the organic solvent S1 by a pulley located at the other end of the first solvent tank 3, and is then wound around a winding roller 11.

At this time, by setting the winding rate of the winding roller 11 to a faster rate than the discharge rate from the nozzle 7, the spun solubilized collagen fibers F are stretched while solidifying, and form narrow fibers having an average fineness of 10 dtx or less.

During the period required for the collagen fibers to solidify, or specifically during the period required for the exterior of the collagen fibers to solidify, spinning and drawing of the collagen fibers is performed. During this period, the collagen fibers exist within the organic solvent, and therefore the water content within the collagen fibers is substituted with the organic solvent.

The time required for solidification varies depending on the fineness and the like of the fibers undergoing spinning. Considering such factors, the time required for solidification of the solubilized collagen fibers is generally set to approximately 8 seconds.

If a value of approximately 5 m/minute is used for the winding rate of the winding roller 11, then the length of the first solvent tank 3 in the direction of operation must be approximately 70 cm or longer.

By discharging the solubilized collagen aqueous solution through the nozzle and into the organic solvent, the solubilized collagen can be spun.

This spinning can be achieved by using a device such as a nozzle or shower head which has discharge holes that can discharge a fluid in a fiber-like form and can therefore disperse and release the fluid. The solubilized collagen aqueous solution, which has a solubilized collagen concentration from 2 to 10 mass %, and preferably from 3 to 7 mass %, is discharged into the organic solvent at a rate of 20 to 500 g/minute, and preferably 30 to 150 g/minute, through a dispersion release device having a hole diameter of approximately 0.05 to 1 mm, and preferably approximately 0.05 to 0.3 mm. As a result, solubilized collagen fibers having an average fineness of approximately 10 to 100 dtx (measured at 20° C. and 65% RH using a fineness meter) can be formed.

The thickness of the solubilized collagen fibers can be narrowed by adjusting the concentration of the discharged solubilized collagen aqueous solution, or by appropriate selection of the hole diameter of the discharge nozzle. If the concentration of the solubilized collagen aqueous solution is too low, then the spun fibers tend to rupture more easily, and a powder-like solid tends to be produced. If the nozzle hole diameter is too narrow, then the flow resistance increases, and an excessively large discharge pressure occurs at the nozzle. Consequently, the collagen fibers discharged from the nozzle in a free state undergo shrinkage in the fiber length direction during solidification, shrinking to less than approximately 0.6 times the original length at the time of discharge and resulting in an increase in the fineness.

There is a limit to how far the fineness can be reduced using the methods of narrowing the nozzle hole diameter and reducing the concentration of the solubilized collagen aqueous solution.

In one method of resolving this problem, the collagen fibers that are spun in the solvent can be wound at a rate that is at least approximately 0.6 times the discharge rate. By using this method, the pulling force applied to the collagen fibers during spinning acts against the shrinkage in the fiber length direction, thereby drawing the fibers and enabling the preparation of fine fibers of 10 dtx or less.

However, if the winding rate is too fast, then the fibers are prone to rupture, and therefore drawing is performed with the ratio of the winding rate relative to the discharge rate (namely, the draft) adjusted to a value of 1.5 or less.

Considering the above factors, ideal conditions for spinning collagen fibers having an average fineness of 10 dtx or less include a collagen aqueous solution concentration of 3 to 7 mass %, and preferably 3.5 to 5 mass %, a nozzle hole diameter of 0.05 to 0.18 mm, and preferably approximately 0.09 to 0.11 mm, and a draft of at 0.6 to 1.5, and preferably 1.0 to 1.2.

Each of the various conditions may be set within the respective range described above, in accordance with a formula I shown below.

T=100˜r²cd/D Formula 1

In this formula, T represents the fineness (dtx), r represents the nozzle hole radius (mm), c represents the collagen aqueous solution concentration (mass %), d represents the collagen specific gravity (g/ml), and D represents the draft.

In terms of the types of numerical values that are actually employed, setting the various values so as to achieve a discharge rate of approximately 2 to 7 m/minute and a winding rate of approximately 2 to 10 m/minute is practical.

The wound solubilized collagen fibers are dried in a sterilized environment by air drying using sterilized air. This removes any residual water. In the case of the types of fine fibers produced in the present invention, if the fibers are in mutual contact and drying is performed in this state, then the fibers tend to adhere and bond together, forming a fibrous lump.

The reason for this observation is that because the organic solvent is removed first during drying, the residual water content contained within the solubilized collagen fibers re-dissolves the solidified collagen, and therefore as the fibers become finer, adhesion of the fibers becomes more significant.

In order to prevent this adhesion, in the present invention, the solubilized collagen fibers are immersed in a hydrophilic organic solvent prior to drying. By bringing the fibers into contact with the hydrophilic organic solvent, the water content within the collagen fibers diffuses within the organic solvent and is replaced with the organic solvent, meaning the water content of the fibers decreases and the organic solvent content increases. As a result, adhesion of the fibers during drying is reduced.

The water content of the hydrophilic organic solvent used for immersion must be low, and specifically, an organic solvent having a water content of 5 mass % or less is typically used. Specific examples of hydrophilic organic solvents that can be used include alcohols such as methanol, ethanol, and isopropanol, and acetone. A mixed solvent containing a plurality of these types of solvents may also be used. In order to avoid the retention of only water during the drying of the collagen fibers, the use of a solvent having a boiling point close to that of water, or a solvent that undergoes azeotropic distillation with water is effective, and specific examples of such solvents include ethanol and isopropanol.

When the spun solubilized collagen fibers are immersed in the hydrophilic organic solvent, the water content of the hydrophilic organic solvent increases. Once the organic solvent has been used for repeated immersion treatments, and the water content of the solvent has become excessive, the organic solvent must be replaced. Subjecting the solubilized collagen fibers to a light compression or centrifugal dewatering treatment to reduce the amount of liquid contained within the fibers immediately prior to immersion in the organic solvent is effective in reducing the frequency with which the immersion organic solvent must be replaced.

If a value of approximately 5 m/minute is used for the winding rate of the winding roller 11, then the length of the first solvent tank 3 in the direction of operation must be approximately 70 cm or longer.

(iii) A step of drying the obtained spun and drawn solubilized collagen fiber bundle is conducted as follows.

A drying apparatus used in the present invention is illustrated in FIG. 3.

Prior to performing continuous drying of the spun and drawn solubilized collagen fiber bundle, the solubilized collagen fiber bundle is passed between nip rollers 31 to squeeze a portion of the water content and alcohol content from the fiber bundle, thereby reducing the water content and the alcohol content within the fiber bundle, and this is an important operation in the drying step.

Following passage between the nip rollers 31, the solubilized collagen fiber bundle is introduced into a drying tube (tube-shaped body) 32.

By using a drying tube, the portion through which the air used for drying passes can be controlled from the surrounding environment, and therefore the desired drying treatment can be performed efficiently. The air used for the drying treatment, which passes through the air supply device 33, is sterilized, and exists in a dry state at a temperature of 30° C. or lower, and from the viewpoint of stability is preferably at a temperature of 20° C. or lower, passes through the filter 34 and the air supply unit 39, and is supplied to the drying tube (tube-shaped body) 32. If the tube-shaped drying device 32 is constructed in the form of an aspirator, then the collagen fibers can be fed into the drying device from the suction port. Another effective method involves using a commercially available air gun designed for suctioning and transporting powders and granules (such as the air guns MAG-22S, MAG-22SV, MAG-22L and MAG-22LV manufactured by Trusco Nakayama Corporation, details of the structures of which are included in the operating manuals available from the manufacturer), and feeding the collagen fibers through the suction port.

The air is supplied at a temperature within a range from 30° C. to 0° C. If the temperature exceeds 30° C., then there is a concern that the solubilized collagen may undergo denaturation. Further, if the temperature is lower than 0° C., then the drying efficiency deteriorates.

The humidity must be RH 70% or less. If the humidity exceeds 70%, then the fibers are more likely to undergo agglutination. There are no disadvantages associated with a low humidity.

The solubilized collagen fibers moving through the inside of the drying tube move at a rate of 2 m/h to 3 m/h. The solubilized collagen fibers are pushed by the sterilized air and move though the drying tube.

The actual rate of this movement is dependent on the feed rate through the nip rollers. By appropriate control of the combination of this feed rate and the air flow rate, treatment can be conducted under ideal drying conditions (namely, conditions which not only yield favorable drying, but also result in the production of fibers with an appropriate level of crimping, and minimal adhesion and twisting of the fibers).

When a polyethylene tube having a diameter of 19 mm and a length of 3 m is used, performing drying under conditions including a collagen feed rate of 2 to 3.5 m/minute and an air flow rate of 200 to 300 L/minute yields dry solubilized collagen fibers having minimal fiber agglutination and having a favorable wave applied thereto.

(a) Typical analysis results for the composition of the solubilized collagen fiber bundle are as follows.

Prior to supply to the nip rollers, the solubilized collagen fiber bundle and the like has a solid fraction concentration of 15 to 25 wt %, and a residual alcohol concentration of 70 to 80 wt %.

Following passage through the nip rollers, the solubilized collagen fiber bundle and the like has a solid fraction concentration of 27 to 35 wt %, and a residual alcohol concentration of 65 to 68 wt %.

At the outlet from the drying tube, the solubilized collagen fiber bundle and the like has a solid fraction concentration of 85 to 88 wt %, and a residual alcohol concentration of 1.0 to 6.0 wt %.

(b) In another example, analysis results for the composition of the solubilized collagen fiber bundle are as follows.

Prior to supply to the nip rollers, the solubilized collagen fiber bundle and the like has a solid fraction concentration of 18 to 22 wt %, and a residual alcohol concentration of 75 to 77 wt %.

Following passage through the nip rollers, the solubilized collagen fiber bundle and the like has a solid fraction concentration of 27 to 32 wt %, and a residual alcohol concentration of 66 to 67 wt %.

At the outlet from the drying tube, the solubilized collagen fiber bundle and the like has a solid fraction concentration of 82 to 87 wt %, and a residual alcohol concentration of 1.0 to 6.0 wt %.

By subjecting the solubilized collagen fiber bundle at the tube outlet to a further drying operation, the residual alcohol concentration can be reduced to 0.01 wt % or less. If the solubilized collagen fibers are processed, then these properties do not change, meaning these numerical values represent the values for the solubilized collagen fibers.

Because the bundle of the solubilized collagen fibers F is dried without application of a pulling load, a fiber bundle composed of crimped solubilized collagen fibers can be obtained. Because there are almost no portions where the fibers have adhered to one another, making dissolution difficult, the speed of dissolution is improved dramatically compared with fiber bundles obtained using conventional batch drying methods, and is adequate to enable the fiber bundle to be used in a cosmetic material prepared immediately prior to use.

Moreover, by performing appropriate fiber opening of the dried solubilized collagen fiber bundle, a fibrous state solubilized collagen can be obtained.

Following completion of the drying operation, the solubilized collagen fiber bundle is subjected to fiber opening. Specifically, the fiber bundle is supplied continuously to an automatic fiber opening device, and is converted to a fibrous state by hitting and disentangling the fiber bundle with wire drums. The fibers that constitute the solubilized collagen fiber bundle are broken up by the wire drums of the automatic fiber opening device to form fibers having a length of 1 to 20 cm (average: 7 to 8 cm). Provided the average length of the fibers is at least 2.5 cm, the fibers still undergo entanglement, and by subjecting the dried fiber bundle to continuous fiber opening using the automatic fiber opening device, solubilized collagen fibers having an appropriate length and suitable entanglement properties can be obtained. Open fibrous-state solubilized collagen fibers are discharged from the automatic fiber opening device in the form of a sheet having a uniform density, and can be used as the targeted solubilized collagen fibers. These solubilized collagen fibers can also be used favorably as a cosmetic material prepared immediately prior to use.

In the step for performing the aforementioned fiber opening and producing the targeted solubilized collagen fibers, the fiber opening is performed in the manner described above, and then a grasping device is used to collect the required amount of fibers from the solubilized collagen fiber sheet. Tweezers or the like can be use as the grasping device.

(2) The method for producing solubilized collagen fibers using an enzyme is described below.

The step (i) described in relation to the above production method (1) performed under alkaline conditions may be performed with the conditions altered as follows.

A step of decomposing insoluble collagen fibers using a protein degrading enzyme (protease) and extracting a solubilized collagen aqueous solution, and a step of performing pH modification to prepare a solubilized collagen aqueous solution that functions as a solubilized collagen fiber raw material.

Namely, a protein containing insoluble collagen is decomposed using a protein degrading enzyme to obtain a collagen-containing product having an isoionic point of 7 to 8, an alkali is added to adjust the pH to 9 to 10, the solubilized collagen is succinylated using a carboxylic acid anhydride to reduce the isoionic point to 5 or less, and the solubilized collagen is precipitated and isolated. In order to obtain a solubilized collagen aqueous solution, an alkali is added in the presence of a buffer to adjust the pH to a value within a range from 6.0 to 7.5 which is greater than the isoionic point.

The step (ii) in the above method (1) of subjecting the solubilized collagen aqueous solution that functions as the solubilized collagen fiber raw material to spinning and drawing to produce a solubilized collagen fiber bundle is also performed in the method (2) for producing solubilized collagen fibers using a protein degrading enzyme, and is performed in the same manner as that described above for step (ii) in the method (1).

Namely, the solubilized collagen aqueous solution is discharged into an organic solvent in a thread-like form, the solubilized collagen is spun into a fiber bundle, the spun solubilized collagen fiber bundle is drawn by winding, and the drawn solubilized collagen fiber bundle is then immersed in a hydrophilic organic solvent.

The step (iii) in the above method (1) of drying the solubilized collagen fiber bundle, and then performing fiber opening of the solubilized collagen fiber bundle to produce solubilized collagen fibers for use in cosmetics is also performed in the method (2) for producing solubilized collagen fibers using an enzyme, and is performed in the same manner as that described above for step (iii) in the method (1).

Namely, the solubilized collagen fiber bundle is dried by passing the above solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower into the tube, using the moving bed of air to move and dry the solubilized collagen fiber bundle inside the tube, and then extracting the solubilized collagen fiber bundle from the tube. The dried solubilized collagen fiber bundle is then subjected to fiber opening to produce the targeted solubilized collagen fibers.

Methods for producing the solubilized collagen fiber bundle of the present invention can be classified as one of the following two methods.

(1) A solubilized collagen fiber bundle obtained via a method for producing solubilized collagen fibers comprising (i) a step of subjecting a product obtained by decomposing a skin sample containing insoluble collagen fibers under alkaline conditions to a neutralization and desalting treatment, separating a neutralized and desalted skin sample, and subsequently extracting a solubilized collagen aqueous solution having an isoionic point of pH 5.0 or less, and a step of adjusting the pH of the solubilized collagen aqueous solution in the presence of a buffer salt to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle from (ii) above by passing the solubilized collagen fiber bundle through nip rollers, introducing the thus obtained solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of subsequently cutting the fiber bundle to produce the targeted solubilized collagen fiber bundle.

(2) A solubilized collagen fiber bundle obtained via a method for producing solubilized collagen fibers comprising (i) a step of decomposing a skin sample containing insoluble collagen fibers using a protein degrading enzyme (protease) and extracting a solubilized collagen aqueous solution having an isoionic point of 7 to 8, and a step of adding an alkali to the solubilized collagen aqueous solution to adjust the pH to a value of 9 to 10, using a carboxylic acid anhydride to succinylate the solubilized collagen and reduce the isoionic point to 5 or less, subsequently precipitating and separating the solubilized collagen, and then adding an alkali in the presence of a buffer salt to adjust the pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower into the tube, using the moving bed of air to move and dry the solubilized collagen fiber bundle inside the tube, and then extracting the solubilized collagen fiber bundle from the tube, and a step of cutting the fiber bundle to produce the targeted solubilized collagen fiber bundle.

Methods for producing the solubilized collagen fiber ball of the present invention can be summarized as follows.

(1) The operation for producing the solubilized collagen fiber ball is as follows.

The fiber ball, which is prepared by forming a solubilized collagen fiber bundle in which the composition is distributed uniformly throughout the fibers into a spherical shape, is produced by expanding the solubilized collagen fiber bundle, and moving the fiber bundle along a cylindrical opening sandwiched between a cylindrical inner surface and a helical groove that rotates around the inside of this cylindrical inner surface at a rate of 50 to 150 revolutions per minute, so that the fiber bundle moves along the helical groove at a rate of 30 to 100 cm/second.

(2) The targeted solubilized collagen fibers are produced by a method comprising (i) a step of subjecting a product obtained by decomposing a skin sample containing insoluble collagen fibers under alkaline conditions to a neutralization and desalting treatment, separating a neutralized and desalted skin sample, and subsequently extracting a solubilized collagen aqueous solution having an isoionic point of pH 5.0 or less, and a step of adjusting the pH of the solubilized collagen aqueous solution in the presence of a buffer salt to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle from (ii) above by passing the solubilized collagen fiber bundle through nip rollers, introducing the thus obtained solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

(3) The step of performing fiber opening to produce the targeted solubilized collagen fibers may also be performed by performing fiber opening to produce a fibrous state, and then using a grasping device to collect the required amount of fibers.

(4) The solubilized collagen fiber ball is produced by a method comprising (i) a step of decomposing a skin sample containing insoluble collagen fibers using a protein degrading enzyme (protease) and extracting a solubilized collagen aqueous solution having an isoionic point of 7 to 8, and then adjusting the pH of the solubilized collagen aqueous solution in the presence of a buffer to a value within a range from 6.0 to 7.5, thus preparing a solubilized collagen aqueous solution that functions as a solubilized collagen fiber raw material, wherein this step is performed by decomposing a protein containing insoluble collagen using a protein degrading enzyme to obtain a collagen-containing product with an isoionic point of 7 to 8, adding an alkali to adjust the pH to a value of 9 to 10, using a carboxylic acid anhydride to succinylate the solubilized collagen and reduce the isoionic point to 5 or less, subsequently precipitating and separating the solubilized collagen, and then adding an alkali in the presence of a buffer to adjust the pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in (i) above into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from (ii) above by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of RH 70% or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

(5) The step of performing fiber opening to produce the targeted solubilized collagen fibers may also be performed by performing fiber opening to produce a fibrous state, and then using a grasping device to collect the required amount of fibers.

A fiber ball production apparatus is used in the methods for producing fiber balls with solubilized collagen fibers according to the present invention.

Methods and apparatus for forming a material composed of fibers or the like into a spherical shape are already known (such as JP 2001-295170 A, JP 3,601,004 B and JP 10-266051 A). Moreover, fiber balls prepared by forming absorbent cotton into a spherical shape are also known as medical items (such as JP 3,557,587 B), and these methods and apparatus can be used with appropriate setting of the conditions.

As described above, conventional fiber ball apparatus can be improved for use as the fiber ball production apparatus, but the inventors of the present invention also developed and tested a novel cylindrical fiber ball production apparatus and flat plate-shaped fiber ball production apparatus, and used these apparatus for producing the fiber balls with solubilized collagen fibers.

The properties of the fiber balls with solubilized collagen fibers are as described above.

### EXAMPLES

The present invention is described below in further detail based on a series of examples.

### Example 1

Samples of solubilized collagen fibers for use in cosmetics were prepared in the manner described below, and the time required for dissolution was measured. The isoionic point of the solubilized collagen fibers was confirmed in the manner described below.

Measurement of Isoionic Point

A cationic exchange resin (Amberlite IPR-120B, manufactured by Organo Corporation) and an anionic exchange resin (Amberlite IPA-400, manufactured by Organo Corporation) that had been activated and washed in advance were mixed in a ratio of 2:5 to prepare a mixed bed ion exchanger. Subsequently, 100 ml of this mixed ion exchanger was brought to equilibrium using deionized water, 50 ml of a sample solution prepared with a protein concentration of 5% was added to the ion exchanger, and the mixture was held at 40° C. in a water bath and stirred gently for 30 minutes. The supernatant was then removed from the mixture, the pH of the supernatant was measured, and this measured value was used as the isoionic point (see the method described by J. W. Janus, A. W. Kenchington and A. G. Ward, Research, Vol. 4, 247-248 (1951)).

### Example 2

### Sample 1

Preparation of Solubilized Collagen Aqueous Solution Under Alkaline Conditions

Using wet-salted pig hide as a raw material, liming was performed. Specifically, one half of a single wet-salted pig hide (approximately 4 kg) was cut into small skin pieces approximately 3 cm square, an amount of water equivalent to 300% of the mass of the skin pieces and 0.6% of a nonionic surfactant were added and stirred to wash the skin pieces, and the skin pieces were then recovered. Subsequently, the skin pieces were combined with an amount of water equivalent to 300% of the mass of the skin pieces, together with 0.6% of a nonionic surfactant and 0.75% of sodium carbonate, the mixture was stirred for 2 hours, and the skin pieces were once again recovered. Next, the recovered skin pieces were washed twice with amounts of water equivalent to 700% of the mass of the skin pieces, and the skin pieces were then combined with an amount of water equivalent to 300% of the mass of the skin pieces, together with 0.15% of a nonionic surfactant, 3.6% of sodium hydrosulfide, 0.84% of sodium sulfide and 2.4% of calcium hydroxide, the mixture was stirred for 16 hours, and the skin pieces were once again recovered and washed three times with amounts of water equivalent to 700% of the mass of the skin pieces.

Next, 8,000 g of an aqueous solution was prepared containing 6 mass % of sodium hydroxide, 15 mass % of sodium sulfate and 1.25 mass % of monomethylamine, and then 2,000 g of the above skin pieces (a dried mass of approximately 500 g) were added to the solution and stirred thoroughly.

The resulting mixture was held inside a sealed container at 25° C., and incubated for 5 days to solubilize the collagen. With the aqueous solution undergoing gentle stirring, an amount of sulfuric acid equal to the amount of alkali within the aqueous solution was added dropwise to neutralize the solution, thereby adjusting the pH to 4.8.

Following neutralization, the skin pieces were removed and pressed to remove any liquid contained therein, and the skin pieces were subsequently stirred for 30 minutes in approximately 8,000 g of an aqueous solution of lactic acid with a pH of 5.0, and then pressed and dewatered. This operation was repeated a further 4 times to achieve satisfactory desalting. In a neutralized state, the pH of the skin pieces is close to the isoionic point of the solubilized collagen, and therefore although the collagen is solubilized, it undergoes almost no dissolution in water even during the desalting operations, but is rather retained within the skin pieces.

The collagen content of the skin pieces following desalting was calculated from the results of measuring the total nitrogen content using the Kjeldahl method, and based on this calculated collagen content value, a sample of the desalted skin pieces equivalent to 120 g of collagen was mixed thoroughly with water and sodium lactate to obtain an aqueous solution having a collagen concentration of 4.4 mass % and a sodium lactate concentration of 1.2 mass %, thus yielding 4,000 g of a solubilized collagen aqueous solution. Subsequently, a small amount of a 20% aqueous solution of sodium hydroxide was added and stirred to adjust the pH to a value of 6.7.

Production of Solubilized Collagen Fibers

The tank 5 of a production apparatus 1 having the structure illustrated in FIG. 1 was charged with 4,000 g of the solubilized collagen aqueous solution obtained above, and 18 L of isopropanol was used as the organic solvent and placed in the first solvent tank 3 having a length of 3 m and a width of 10 cm. By operating the gear pump 9, the solubilized collagen aqueous solution was discharged into the organic solvent from the discharge holes of the nozzle 7, which were directed in the horizontal direction (hole diameter: 0.10 mm, number of holes: 1,000), at a rate of 38 g/minute (discharge rate: 4.8 m/minute). The bundle of solubilized collagen fibers that had undergone spinning within the isopropanol was pulled from the tank by the winding roller 11 at a winding rate of 5 m/minute, and was then immersed in the second solvent tank 13 which contained 5.0 L of isopropanol.

Drying Step

(i) When performing continuous drying of the spun solubilized collagen fiber bundle prepared under the conditions prescribed above, the solubilized collagen fiber bundle was dried without agglutination by passing the fiber bundle through nip rollers, introducing the resulting solubilized collagen fiber bundle having a reduced water content and a reduced alcohol concentration into a drying tube, forming a moving bed of air inside the tube by blowing sterilized air having a temperature of 20° C. and a humidity of 55% RH into the tube (at 238 L/minute), using the moving bed of air to move the solubilized collagen fiber bundle through the inside of the tube at a rate of 3.5 m/minute, thus drying the fiber bundle, and then extracting the solubilized collagen fiber bundle extracted from the tube.

The results of measuring the composition of the solubilized collagen fiber bundle at various stages within the drying treatment step were as follows.

Prior to supply to the nip rollers, the solubilized collagen fiber bundle and the like had a solid fraction concentration of 21 wt %, and a residual alcohol concentration of 76 wt %.

Following passage through the nip rollers, the solubilized collagen fiber bundle and the like had a solid fraction concentration of 29 wt %, and a residual alcohol concentration of 67 wt %, and at the outlet from the drying tube, the solubilized collagen fiber bundle and the like had a solid fraction concentration of 86 wt %, and a residual alcohol concentration of 3.0 wt %.

(ii) In another example, with the exception of altering the nip roller feed rate to 2 m/minute, drying was performed under the same conditions as those described in (i) above. The results of measuring the composition of the solubilized collagen fiber bundle at various stages within the drying treatment step were as follows.

Prior to supply to the nip rollers, the solubilized collagen fiber bundle and the like had a solid fraction concentration of 20 wt %, and a residual alcohol concentration of 74 wt %.

Following passage through the nip rollers, the solubilized collagen fiber bundle and the like had a solid fraction concentration of 30 wt %, and a residual alcohol concentration of 66 wt %.

At the outlet from the drying tube, the solubilized collagen fiber bundle and the like had a solid fraction concentration of 87 wt %, and a residual alcohol concentration of 1.5 wt %.

Using an automatic fiber opening device, the obtained solubilized collagen fiber bundle was subjected to fiber opening to form solubilized collagen fibers.

The obtained solubilized collagen fibers were composed of 81 wt % of solubilized collagen, 3 wt % of sodium lactate, 1.5 wt % of isopropyl alcohol, 11.5 wt % of water, and 3 wt % of ash (totaling 100 wt %), had an average fineness of 6 dtx and a length of 10 to 35 mm, and had a wavy shape.

Using a fiber ball production apparatus, the above solubilized collagen fibers were used to produce fiber balls with solubilized collagen fibers.

A summary of the production apparatus conditions is presented below.

A fiber ball forming apparatus manufactured by Ikegami Seisakusho Co., Ltd. was used. The length of the groove (from the inlet to the outlet) was 1,980 cm, the width of the groove for the first 180 cm from the inlet was 16 mm, and the groove width then narrowed to 15 mm for the next 120 cm, 14 mm for the next 60 cm, and then 13 mm for the 1,620 cm of groove length closest to the outlet.

In order to obtain the intended rate of movement of the fiber balls, an appropriate frictional resistance is required between the cylinder and the fiber balls, and therefore a resin tape that had undergone non-slip processing was adhered to the inner surface of the cylinder. By using this method, the intended rate of movement was achieved, whereas no foreign matter fell from the product as a result of the friction with the fiber balls of solubilized collagen fibers. The rate of rotation of the helical groove was set to 108 revolutions per minute, and the solubilized collagen fibers moved through the helical groove at a rate of 72 cm/second.

Fifteen mg of the above solubilized collagen fibers were weighed using an electronic balance and supplied to the fiber ball production apparatus, and fiber balls were then produced under the conditions described above. The obtained fiber balls had a diameter of 15 mm.

The fiber balls with solubilized collagen fibers were placed in a container having a structure in which a container portion 43 in which the solubilized collagen fiber ball is disposed, and a container portion 44 for holding the water in which the solubilized collagen fiber ball is dissolved are integrated within a single container, wherein the container portion in which the solubilized collagen fiber ball is disposed has a structure 45 in which the side wall of the container is formed as a curved surface that narrows toward the bottom of the container, a horizontal flange 46 is formed at the top of the container, and the top of the flange is sealed with a top cover sheet 41 (see FIG. 4).

The material of the container main body was a material that blocks light and prevents the transmission of oxygen and moisture, and an example of the material that was used is PBP (a material in which an ethylene-vinyl alcohol copolymer resin (EVOH) is sandwiched between polypropylene chains). The shape of the container was a structure having a length of 30 mm, a width of 25 mm and a depth of 20 mm. The material for the cover employed a structure in which a PET layer was laminated on top of aluminum. The cover was sealed by fusion to the container main body.

A solubilized collagen fiber ball having a mass of 10 mg was stored in each container.

The fiber balls were stored at 25° C. for 50 days.

One cc of water was added to the container, and when mixing was performed with the index finger, the fibers dissolved instantly and uniformly in 20 seconds.

### Example 3

Preparation of Aqueous Liquid for Cosmetics

An aqueous liquid (with a pH of approximately 6.6) for cosmetics was prepared from 5.0 mass % of 1,2-pentanediol, 5.0 mass % of glycerol, 3 mass % of 1,3-butylene glycol, 0.66 mass % of sodium citrate, 0.03 mass % of citric acid, with the remainder composed of sterilized water.

A 10 mg collagen sample (as shown below in Table 1) was placed in the palm of the hand, one cc of the above aqueous liquid was added, the mixture was blended with a finger to achieve dissolution, and the time required to completely dissolve the collagen sample was measured. For each sample, the measurement was repeated 5 times, and the minimum value, the maximum value, the mean value and the median value were determined from the measurement results. The results are shown in Table 1.

The samples were a solubilized collagen fiber bundle 1 of the present invention, fiber balls with solubilized collagen fibers of the present invention (3 examples), and conventional solubilized collagen fibers (comparative example).

TABLE 1
Time required for dissolution of fiber balls with solubilized
collagen fibers (seconds) Water temperature (25° C.)

| | Minimum | Maximum | | Median |
|---|---|---|---|---|
| Sample | value | value | Mean value | value |
| Collagen fiber bundle 1 | 27 | 35 | 30 | 29 |
| Fiber ball 1 | 8 | 11 | 10 | 10 |
| Fiber ball 2 | 12 | 15 | 13 | 13 |
| Fiber ball 3 | 14 | 17 | 16 | 16 |
| Comparative example 1 | 65 | 94 | 82 | 85 |

Collagen fiber bundle 1: the case in which a collagen fiber bundle of the present invention was dissolved. This result represents the case where the fibers were not formed into a fiber ball, but rather 1 cm of the collagen fiber bundle (weight: 10 mg) was used for the dissolution.

Fiber ball 1: a solubilized collagen fiber ball obtained using the present invention, having a diameter of 3 mm, a weight of 5 mg, and an average fineness of 3 dtx.

Fiber ball 2: a solubilized collagen fiber ball obtained using the present invention, having a diameter of 10 mm, a weight of 10 mg, and an average fineness of 7 dtx.

Fiber ball 3: a solubilized collagen fiber ball obtained using the present invention, having a diameter of 25 mm, a weight of 20 mg, and an average fineness of 10 dtx.

Comparative example 1: the case in which conventional collagen fibers produced in accordance with JP 2006-342472 A were used (namely, fibers produced using a conventional collagen fiber production method in which the drying step was performed by suspended air drying inside a clean bench, whereas the other steps were the same as those of the present invention).

For all of the fiber balls 1, 2 and 3 with solubilized collagen fibers, the mean value and median value for the dissolution time was within a range from 10 to 13 seconds. The corresponding dissolution times for the collagen fiber bundle of the present invention were 30 seconds and 29 seconds. The results for the fiber balls 1, 2 and 3 with solubilized collagen fibers were very good. These results were able to satisfactorily confirm the effects of the fiber balls. Further, even for the collagen fiber bundle, although the results did not approach those of the fiber balls with solubilized collagen fibers, they were still sufficiently favorable to enable use as a cosmetic material prepared immediately prior to use.

The corresponding results for the case where conventional collagen fibers produced in accordance with JP 2006-342472 A were used were 82 seconds and 85 seconds respectively, indicating that a significantly longer time was required compared with the collagen fiber bundle and the fiber balls 1, 2 and 3 of the present invention.

In comparative example 1, portions of agglutinated fibers existed, and although those portions free of agglutination dissolved comparatively quickly, the agglutinated portions formed lumps that required considerable time to dissolve, resulting in an overall dissolution time that was considerably slower than the collagen fibers and fiber balls of the present invention.

### Example 4

### Sample 2

Preparation of Solubilized Collagen Aqueous Solution

A wet-salted pig hide was cut into pieces and limed in the same manner as that described for the sample 1. The thus obtained skin pieces were fed through a chopper having a hole diameter of 16 mm, and then converted to a paste form using a grinding mill (Masscolloider, manufactured by Masuko Sangyo Co., Ltd.). The paste-like pig skin was subjected to a delipidation treatment using ethanol, and was then dried. A 100 g sample was taken from the dried product, 1,900 g of deionized water was added, and hydrochloric acid was added to adjust the pH to 3.0 while the mixture was stirred with a mixer. Subsequently, 20 g of an acidic protease formulation (Denapsin 2P, manufactured by Nagase ChemteX Corporation) was added, and stirring was continued for 24 hours at 25° C. to solubilize the collagen. The thus obtained solubilized collagen aqueous solution was adjusted to a pH of 9 to 10 by adding 2N sodium hydroxide, 40 g of succinic anhydride was dissolved in acetone and added, and with the temperature held at 10° C. and the pH maintained between 9 and 10, reaction (succinylation) was performed for 2 hours. Following completion of the reaction, hydrochloric acid was used to adjust the pH of the reaction solution to 4.5, thus precipitating the collagen.

The precipitate was collected by performing a centrifugal separation at 3,000 G for 10 minutes, and the precipitate was then washed with ethanol and dried, yielding a succinylated solubilized collagen dried product. To a 60 g sample of this dried product were added 29 g of sodium lactate and 1,920 g of water, and the mixture was stirred to obtain a solubilized collagen aqueous solution having a collagen concentration of 4.5 mass % (pH: 6.8, sodium lactate concentration: 1.2 mass %).

Production of Solubilized Collagen Fibers

Using the above solubilized collagen aqueous solution, solubilized collagen fibers were produced using the same apparatus and the same operations as those described for sample 1, yielding 50 g of a solubilized collagen fiber bundle having an average fineness of 4.1 dtx (isoionic point: pH 4.5). The pH of a 0.5 mass % solution prepared by dissolving these solubilized collagen fibers in deionized water was 7.2.

Using collagen samples (shown below in Table 2) obtained using the method for producing solubilized collagen fibers described above, the time required to dissolve each collagen sample was measured in the same manner as that described for example 3. The results are shown in Table 2.

TABLE 2
Time required for dissolution of fiber balls with solubilized collagen
fibers (seconds) Water temperature (25° C.)

| | Minimum | Maximum | | Median |
|---|---|---|---|---|
| Sample | value | value | Mean value | value |
| Collagen fiber bundle 2 | 28 | 34 | 31 | 31 |
| Fiber ball 5 | 12 | 16 | 14 | 14 |

Collagen fiber bundle 2: the case in which a collagen fiber bundle of the present invention was dissolved. This result represents the case where the fibers were not formed into a fiber ball, but rather 1 cm of the collagen fiber bundle (weight: 10 mg) was used for the dissolution.

Fiber ball 5: a solubilized collagen fiber ball obtained using the present invention, having a diameter of 10 mm, a weight of 10 mg, and an average fineness of 7 dtx.

### DESCRIPTION OF REFERENCE SIGNS

1 Production apparatus
3 First solvent tank
5 Piston tank
7 Nozzle
9 Gear pump
11 Winding roller
13 Second solvent tank
S1 Organic solvent
S2 Hydrophilic organic solvent
A Solubilized collagen aqueous solution
F Solubilized collagen fibers
21 Winding device
23 Air flow
25 Hanging device
31 Nip roller
32 Drying tube (tube-shaped body)
33 Air supply device
34 Filter
35 Water collection unit
37 Collagen fibers for cosmetics following drying treatment
38 Storage unit for collagen fibers for cosmetics following drying treatment
39 Air supply unit
41 Top cover sheet
42 Container bottom portion
43 Container portion for solubilized collagen fiber ball
44 Container portion for holding the water for dissolving solubilized collagen fiber ball
45 Portion in which the side wall is formed as a curved surface that narrows toward the bottom of the container
46 Flange that extends outward in a horizontal direction from the top of the container

### INDUSTRIAL APPLICABILITY

Solubilized collagen is being actively used in the fields of foodstuffs and pharmaceuticals. Within these new fields, there is considerable demand for a collagen such as the solubilized collagen fiber ball of the present invention that dissolves instantly and uniformly, and therefore the present invention offers good applicability.

1. A solubilized collagen fiber ball, comprising a fiber bundle with a weight of 3 to 20 mg obtained by cutting solubilized collagen fibers which comprise a composition having a solubilized collagen content of 66 to 87 weight percent, a buffer salt content of 2 to 6 weight percent, a water content of 10 to 22 weight percent and a residual hydrophilic organic solvent content of a trace amount to 6 weight percent (totaling 100 weight percent), have an average fineness of 3 to 10 dtx and a wavy shape, and in which the composition is distributed uniformly along a length direction of the fibers.

2. The solubilized collagen fiber ball according to claim 1, wherein the buffer salt is selected from among sodium citrate, sodium lactate and sodium phosphate.

3. The solubilized collagen fiber ball according to claim 1, wherein the solubilized collagen fibers are obtained by (i) a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting pH of the solubilized collagen aqueous solution in presence of a buffer to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle from the step (ii) by passing the solubilized collagen fiber bundle through nip rollers, introducing a resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

4. The solubilized collagen fiber ball according to claim 1, wherein the solubilized collagen fibers are obtained by (i) a step of decomposing a skin sample containing insoluble collagen fibers using a protein degrading enzyme (protease) and extracting a solubilized collagen aqueous solution having an isoionic point of 7 to 8, and a step of adding an alkali to the solubilized collagen aqueous solution to adjust pH to a value of 9 to 10, using a carboxylic acid anhydride to succinylate the solubilized collagen and reduce the isoionic point to 5 or less, subsequently precipitating and separating the solubilized collagen, and then adding an alkali in presence of a buffer to adjust pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from the step (ii) by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

5. The solubilized collagen fiber ball according to claim 3, wherein the step of performing fiber opening to produce the targeted solubilized collagen fibers is performed by conducting fiber opening to obtain a fibrous state, and then using a grasping device to collect a required amount of fibers.

6. A solubilized collagen fiber ball, comprising a composition having a solubilized collagen content of 66 to 87 weight percent, a buffer salt content of 2 to 6 weight percent, a water content of 10 to 22 weight percent and a residual hydrophilic organic solvent content of a trace amount to 6 weight percent (totaling 100 weight percent), having a diameter of 3 to 25 mm and a weight of 3 to 20 mg, and produced by forming, into a spherical shape, a bundle of solubilized collagen fibers having an average fineness of 3 to 10 dtx, a length of 1 to 20 cm and a wavy shape, in which the composition is distributed uniformly along a length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through an entire interior of the fiber ball in a state that is entangled in some areas and separated in other areas.

7. The solubilized collagen fiber ball according to claim 6, wherein the buffer salt is selected from among sodium citrate, sodium lactate and sodium phosphate.

8. The solubilized collagen fiber ball according to claim 6, wherein the solubilized collagen fibers are obtained by (i) a step of subjecting a product obtained by decomposing a skin sample containing insoluble collagen fibers under alkaline conditions to a neutralization and desalting treatment, separating the neutralized and desalted skin sample, and subsequently extracting a solubilized collagen aqueous solution having an isoionic point of pH 5.0 or less, and a step of adjusting pH of the solubilized collagen aqueous solution in presence of a buffer to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the drawn solubilized collagen fiber bundle in a hydrophilic organic solvent; and (iii) a step of drying the solubilized collagen fiber bundle obtained in the step (ii) by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

9. The solubilized collagen fiber ball according to claim 6, wherein the solubilized collagen fibers are obtained by (i) a step of decomposing a skin sample containing insoluble collagen fibers using a protein degrading enzyme (protease) and extracting a solubilized collagen aqueous solution having an isoionic point of 7 to 8, and a step of adding an alkali to the solubilized collagen aqueous solution to adjust pH to a value of 9 to 10, using a carboxylic acid anhydride to succinylate the solubilized collagen and reduce the isoionic point to 5 or less, subsequently precipitating and separating the solubilized collagen, and then adding an alkali in presence of a buffer to adjust pH to a value within a range from 6.0 to 7.5 which is greater than the isoionic point, thus preparing a solubilized collagen aqueous solution that functions as a solubilized collagen fiber raw material; (ii) a step of discharging the solubilized collagen aqueous solution obtained in the step (i) into an organic solvent in a thread-like form, spinning the solubilized collagen into a fiber bundle, drawing the spun solubilized collagen fiber bundle by winding, and then immersing the solubilized collagen fiber bundle in a hydrophilic organic solvent; and (iii) a step of drying the solubilized collagen fiber bundle from the step (ii) by passing the solubilized collagen fiber bundle through nip rollers, introducing the resulting solubilized collagen fiber bundle having a reduced water content and a reduced concentration of the hydrophilic organic solvent into a drying tube, forming a moving bed of air inside the tube by blowing sterilized air having a temperature of 30° C. or lower and a humidity of 70% RH or lower into the tube, using the moving bed of air to move and dry the solubilized collagen fiber bundle inside the tube, and then extracting the solubilized collagen fiber bundle from the tube, and a step of performing fiber opening to produce the targeted solubilized collagen fibers.

10. The solubilized collagen fiber ball according to claim 8, wherein the step of performing fiber opening to produce the targeted solubilized collagen fibers is performed by conducting fiber opening to obtain a fibrous state, and then using a grasping device to collect a required amount of fibers.

11. The solubilized collagen fiber ball according to claim 8, wherein the fiber ball is formed by supplying the solubilized collagen fibers to an end of a helical groove having a semicircular cross-section provided around a cylindrical outer surface of a cylinder, which is in a rotating state and is provided with a fixed cylindrical cover with a prescribed clearance between the cylinder and the cover, imparting a rotation to the solubilized collagen fibers sandwiched between the rotating helical groove and the fixed cover, moving the solubilized collagen fibers through the helical groove from one end of the cylinder to the other end of the cylinder, and using the rotational force imparted between the groove and the cover during movement through the groove to form the fiber ball.

12. The solubilized collagen fiber ball according to claim 8, wherein the fiber ball is formed using a flat plate-shaped fiber ball production apparatus, which comprises a circular plate that is rotated by a drive device and has a spiral groove with a semicircular cross-section formed in an upper surface that extends from a periphery of the plate toward the center, and a cover that is provided on top of the circular plate with a prescribed clearance therebetween, by supplying solubilized collagen fibers to a solubilized collagen fiber supply port provided at a periphery of the circular plate, imparting rotation to the solubilized collagen fibers sandwiched between the groove and the cover, moving the solubilized collagen fibers through the groove toward the center, and discharging a formed fiber ball from a solubilized collagen fiber outlet provided in the center of the circular plate.

13. A container, wherein a container portion in which a solubilized collagen fiber ball is disposed, and a container portion for holding water in which the solubilized collagen fiber ball is dissolved are integrated within a single container, the container portion in which the solubilized collagen fiber ball is disposed has a structure in which side walls of the container narrow from the top toward the bottom, a horizontal flange is formed at the top of the container, and the top of the flange is sealed with a cover sheet,
wherein the solubilized collagen fiber ball comprises a fiber bundle with a weight of 3 to 20 mg obtained by cutting solubilized collagen fibers which comprise a composition having a solubilized collagen content of 66 to 87 weight percent, a buffer salt content of 2 to 6 weight percent, a water content of 10 to 22 weight percent and a residual hydrophilic organic solvent content of a trace amount to 6 weight percent (totaling 100 weight percent), have an average fineness of 3 to 10 dtx and a wavy shape, and in which the composition is distributed uniformly along a length direction of the fibers.

14. The container according to claim 13, wherein the buffer salt is selected from among sodium citrate, sodium lactate and sodium phosphate.

15. A container, wherein a container portion in which a solubilized collagen fiber ball is disposed, and a container portion for holding water in which the solubilized collagen fiber ball is dissolved are integrated within a single container, the container portion in which the solubilized collagen fiber ball is disposed has a structure in which side walls of the container narrow from the top toward the bottom, a horizontal flange is formed at the top of the container, and the top of the flange is sealed with a cover sheet,
wherein the solubilized collagen fiber ball comprises a composition having a solubilized collagen content of 66 to 87 weight percent, a buffer salt content of 2 to 6 weight percent, a water content of 10 to 22 weight percent and a residual hydrophilic organic solvent content of a trace amount to 6 weight percent (totaling 100 weight percent), has a diameter of 3 to 25 mm and a weight of 3 to 20 mg, and produced by forming, into a spherical shape, a bundle of solubilized collagen fibers having an average fineness of 3 to 10 dtx, a length of 1 to 20 cm and a wavy shape, in which the composition is distributed uniformly along a length direction of the fibers, wherein the fiber ball has a bulk density of 4.0 to 8.0 mg/cm³ and a fiber ball diameter of 3 to 25 mm, and inside the fiber ball, each solubilized collagen fiber is distributed through an entire interior of the fiber ball in a state that is entangled in some areas and separated in other areas.

16. The container according to claim 15, wherein the buffer salt is selected from among sodium citrate, sodium lactate and sodium phosphate.
